(19) United States
(12) Patent Application Publication
(10) Pub. No.: US 2009/0221779 A1
(43) Pub. Date: Sep. 3, 2009

(54) FILM FORMING COMPOSITION, INSULATING FILM, AND ELECTRONIC DEVICE

(75) Inventors: Youhei Kubo, Shizuoka (JP); Haruki Inabe, Shizuoka (JP); Kaoru Iwato, Shizuoka (JP); Katsuyuki Watanabe, Shizuoka (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/392,443

(22) Filed: Feb. 25, 2009

(30) Foreign Application Priority Data
Feb. 29, 2008 (JP) 2008-049995

(51) Int. Cl. C08F 38/00; C07C 15/12; C07C 69/76; C07C 43/20

(52) U.S. Cl. 526/285; 585/25; 560/76; 568/640

(57) ABSTRACT

A film forming composition is provided that includes a compound represented by Formula (1) below and/or a polymer polymerized using at least a compound represented by Formula (1) below

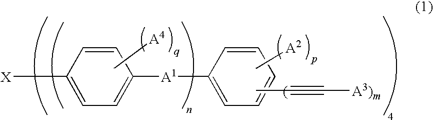

(in Formula (1), each A¹ independently denotes a single bond, —CO—, —NR—, —O—, —SO₂—, —CH═CH—, —C≡C—, or a group in which two or more of the above are combined, A² and A⁴ independently denote a monovalent organic group having 1 to 10 carbons, each A³ independently denotes a hydrogen atom or a monovalent organic group having 1 to 10 carbons but excluding a phenyl group, X denotes a tetravalent organic group having 1 to 9 carbons, R denotes a hydrogen atom or an alkyl group having 1 to 10 carbons, each m independently denotes an integer of 1 to 5, each n independently denotes an integer of 1 or more, each p independently denotes an integer of 0 to 4, and each q independently denotes an integer of 0 to 4). There are also provided a film obtained using the film forming composition and an electronic device having the film.

### BACKGROUND OF THE INVENTION

1. Field of the Invention

The present invention relates to a film forming composition, an insulating film obtained using the film forming composition, and an electronic device having the insulating film.

2. Description of the Related Art

In recent years, in the field of electronic materials, accompanying progress in high integration, multifunctionalization, and high performance, circuit resistance and inter-wiring capacitance have increased, thus causing increases in power consumption and delay time. In particular, since the increase in delay time is the main cause of a decrease in signal speed or the occurrence of crosstalk in a device, in order to reduce the delay time and increase the device speed there is a need to reduce parasitic resistance and parasitic capacitance. As a specific measure for reducing the parasitic capacitance, covering the area around the wiring with a low permittivity interlayer insulating film has been attempted. Furthermore, the interlayer insulating film is required to have excellent heat resistance such that it can withstand a thin film formation step when producing a package substrate or a back end step such as chip connection or pin attachment, or to have excellent chemical resistance such that it can withstand a wet process. Moreover, in recent years Cu wiring, which has low resistance, has been introduced to replace Al wiring; accompanying this, planarization by CMP (chemical mechanical polishing) is commonly carried out, and high mechanical strength that allows the film to withstand this process is needed.

As compounds exhibiting low permittivity, polymers formed from saturated hydrocarbons are generally cited. These polymers have lower molar polarization than polymers formed from a hetero atom-containing unit or an aromatic hydrocarbon unit, and therefore exhibit lower permittivity. However, hydrocarbons having high flexibility such as polyethylene do not have sufficient heat resistance and cannot be used in an electronic device.

In contrast thereto, a polymer having introduced into the molecule adamantane or diamantane, which are saturated hydrocarbons with a rigid cage structure, is disclosed to have low permittivity (JP-A-2003-292878; JP-A denotes a Japanese unexamined patent application publication).

### BRIEF SUMMARY OF THE INVENTION

Polymers in which adamantane or diamantane has been introduced into the molecule as described in JP-A-2003-292878 are usually designed with pores introduced into the film with the aim of giving low permittivity. Because of this there are the problems that the heat resistance and mechanical resistance are degraded or water is adsorbed in the pores. There are cases in which the use of an additive can solve these problems, but there are other problems in that the uniformity or film surface condition of a film are degraded, and these problems need to be solved.

It is an object of the present invention to provide a film forming composition that enables a film having excellent heat resistance and mechanical strength, low permittivity, and good film surface condition to be formed, an insulating film obtained using the film forming composition, and an electronic device having the insulating film.

As a result of an intensive investigation by the present inventors, it has been found that the above problems can be solved by the constitutions of <1>, <9>, or <11> below. They are described below together with <2> to <8>, and <10>, which are preferred embodiments.
<1> A film forming composition comprising a compound represented by Formula (1) below and/or a polymer polymerized using at least a compound represented by Formula (1) below

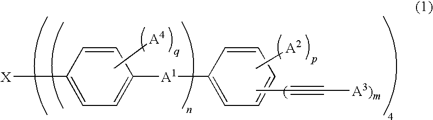

(in Formula (1), each A¹ independently denotes a single bond, —CO—, —NR—, —O—, —SO₂—, —CH═CH—, —C≡C—, or a group in which two or more of the above are combined, A² and A⁴ independently denote a monovalent organic group having 1 to 10 carbons, each A³ independently denotes a hydrogen atom or a monovalent organic group having 1 to 10 carbons but excluding a phenyl group, X denotes a tetravalent organic group having 1 to 9 carbons, R denotes a hydrogen atom or an alkyl group having 1 to 10 carbons, each m independently denotes an integer of 1 to 5, each n independently denotes an integer of 1 or more, each p independently denotes an integer of 0 to 4, and each q independently denotes an integer of 0 to 4),
<2> the film forming composition according to <1> above, wherein it comprises a polymer obtained by thermally polymerizing or by polymerizing in the presence of a radical initiator or in the presence of a transition metal catalyst a monomer represented by Formula (1) above,
<3> the film forming composition according to <1> or <2> above, wherein it comprises a compound having a cage structure and/or a polymer having a cage structure,
<4> the film forming composition according to any one of <1> to <3> above, wherein it comprises a polymer having a cage structure,
<5> the film forming composition according to any one of <1> to <4> above, wherein it comprises a polymer having a cage structure obtained by polymerizing a monomer having a cage structure in the presence of a radical initiator or in the presence of a transition metal catalyst,
<6> the film forming composition according to <5> above, wherein the monomer having a cage structure has a polymerizable carbon-carbon double bond and/or carbon-carbon triple bond,
<7> the film forming composition according to any one of <3> to <6> above, wherein the cage structure is a structure selected from the group consisting of adamantane, biadamantane, diamantane, triamantane, and tetramantane,
<8> the film forming composition according to <5> or <6> above, wherein the monomer having a cage structure is a monomer selected from the group consisting of monomers represented by Formulae (2) to (7) below

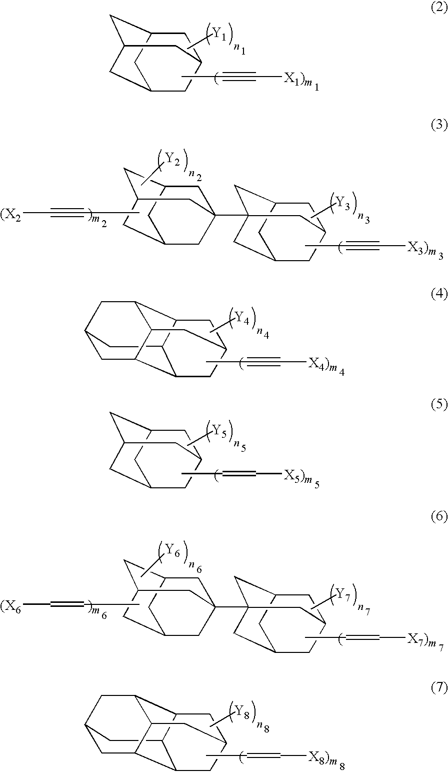

(in Formulae (2) to (7), X₁ to X₈ independently denote a hydrogen atom, an alkyl group, an alkenyl group, an alkynyl group, an aryl group, a silyl group, an acyl group, an alkoxycarbonyl group, or a carbamoyl group, Y₁ to Y₈ independently denote a halogen atom, an alkyl group, an aryl group, or a silyl group, m₁ and m₅ denote an integer of 1 to 16, n₁ and n₅ denote an integer of 0 to 15, m₂, m₃, m₆, and m₇ independently denote an integer of 1 to 15, n₂, n₃, n₆, and n₇ denote an integer of 0 to 14, m₄ and m₈ denote an integer of 1 to 20, and n₄ and n₈ denote an integer of 0 to 19),
<9> a film obtained using the film forming composition according to any one of <1> to <8> above,
<10> the film according to <9> above, wherein it is an insulating film, and
<11> an electronic device comprising the film according to <9> or <10> above.

### BRIEF DESCRIPTION OF DRAWINGS

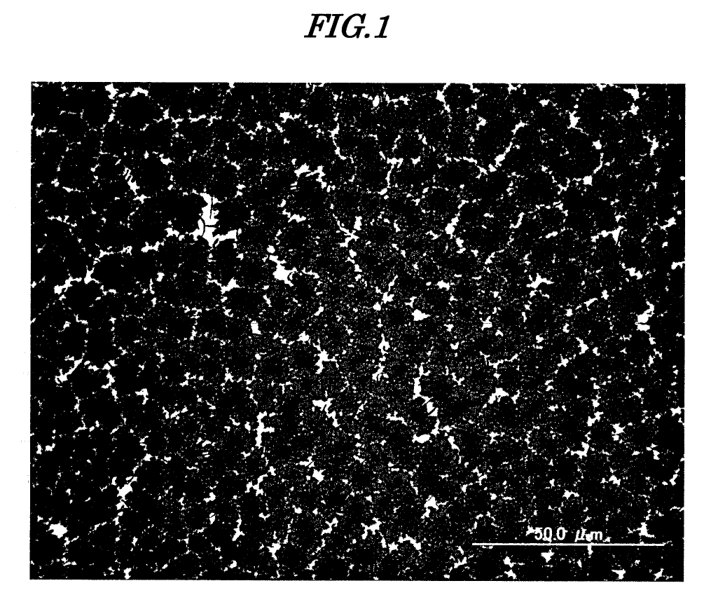

(FIG. 1) A diagram of film surface condition examined using an OLYMPUS MX50 when 30 wt % of the compound below was added to polymer (4).

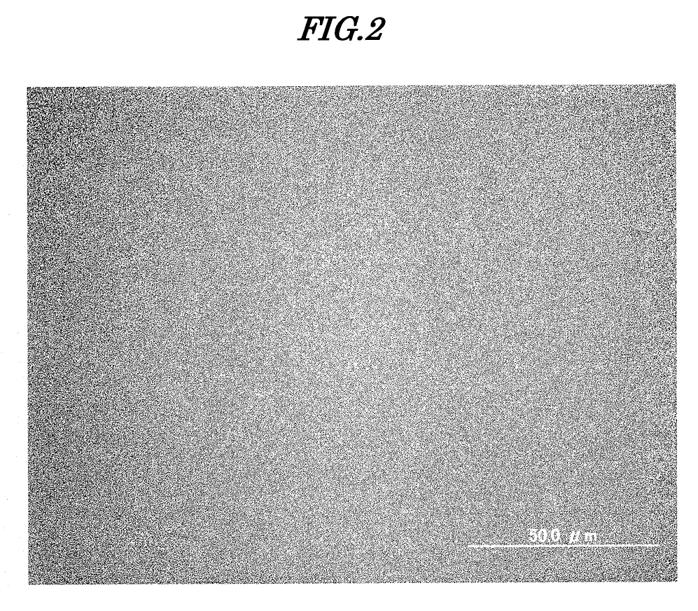

(FIG. 2) A diagram of film surface condition examined using an OLYMPUS MX50 when polymer (4) alone was used.

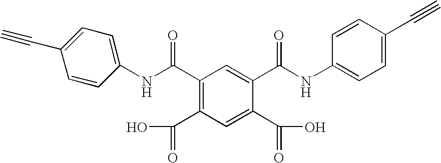

### DETAILED DESCRIPTION OF THE INVENTION

The present invention is explained in detail below.

### Film Forming Composition

The film forming composition (hereinafter, also simply called a ‘composition’) of the present invention comprises a compound represented by Formula (1) below and/or a polymer polymerized using at least a compound represented by Formula (1) below.

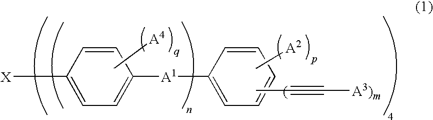

(In Formula (1), each A¹ independently denotes a single bond, —CO—, —NR—, —O—, —SO₂—, —CH═CH—, —C≡C—, or a group in which two or more of the above are combined, A² and A⁴ independently denote a monovalent organic group having 1 to 10 carbons, each A³ independently denotes a hydrogen atom or a monovalent organic group having 1 to 10 carbons but excluding a phenyl group, X denotes a tetravalent organic group having 1 to 9 carbons, R denotes a hydrogen atom or an alkyl group having 1 to 10 carbons, each m independently denotes an integer of 1 to 5, each n independently denotes an integer of 1 or more, each p independently denotes an integer of 0 to 4, and each q independently denotes an integer of 0 to 4.)

### Compound Represented by Formula (1)

The film forming composition of the present invention comprises a compound represented by Formula (1) above and/or a polymer polymerized using at least a compound represented by Formula (1) above.

With regard to the compound represented by Formula (1) above in the film forming composition of the present invention, one type thereof may be used on its own or two or more types thereof may be used in combination.

Each A¹ in Formula (1) above independently denotes a single bond, —CO—, —NR—, —O—, —SO₂—, —CH═CH—, —C≡C—, or a group in which two or more of the above are combined, preferably denotes —CO—O—, —O—CO—, —CO—NR—, —NR—CO—, —O—, —CH═CH—, or —C≡C—, more preferably denotes —CO—O—, —O—CO—, —O—, —CH═CH—, or —C≡C—, and yet more preferably denotes —O— or —C≡C—. The above R denotes a hydrogen atom or an alkyl group having 1 to 10 carbons. The alkyl group may be a straight chain or have a branch or a ring structure.

A² and A⁴ in Formula (1) above independently denote a monovalent organic group having 1 to 10 carbons.

The monovalent organic group having 1 to 10 carbons is not particularly limited, and an alkyl group having 1 to 10 carbons and an aryl group having 1 to 10 carbons can be cited as preferred examples. Furthermore, the alkyl group may be a straight chain or have a branch or a ring structure.

Specific preferred examples of the alkyl group having 1 to 10 carbons and the aryl group having 1 to 10 carbons include a methyl group, an ethyl group, a propyl group, a butyl group, a pentyl group, a hexyl group, a cyclohexyl group, a heptyl group, an octyl group, a phenyl group, and a naphthyl group.

Moreover, the monovalent organic group having 1 to 10 carbons may be a group in which one carbon atom of these groups is replaced by a silicon atom, such as a trialkylsilyl group or a triarylsilyl group. Examples of the trialkylsilyl group include a trimethylsilyl group, a triethylsilyl group, and a tripropylsilyl group, and examples of the triarylsilyl group include a triphenylsilyl group.

Among them, A² and A⁴ are preferably independently a methyl group, an ethyl group, a cyclohexyl group, or a phenyl group, and more preferably a methyl group.

Each A³ in Formula (1) above independently denotes a hydrogen atom or a monovalent organic group having 1 to 10 carbons but excluding a phenyl group. A³ in Formula (1) above is not a phenyl group.

The monovalent organic group, denoted by A³, having 1 to 10 carbons but excluding a phenyl group is not particularly limited, but an alkyl group having 1 to 10 carbons and an aryl group having 1 to 10 carbons but excluding a phenyl group can be cited as preferred examples. The alkyl group may be a straight chain or have a branch or a ring structure.

Specific preferred examples of the alkyl group having 1 to 10 carbons and the aryl group having 1 to 10 carbons but excluding a phenyl group include a methyl group, an ethyl group, a propyl group, a butyl group, a pentyl group, a hexyl group, a cyclohexyl group, a heptyl group, an octyl group, and a naphthyl group.

Furthermore, the monovalent organic group having 1 to 10 carbons may be a group in which one carbon atom of these groups is replaced by a silicon atom, such as a trialkylsilyl group. Examples of the trialkylsilyl group include a trimethylsilyl group, a triethylsilyl group, and a tripropylsilyl group.

Among them, A³ is preferably a hydrogen atom or a trimethylsilyl group, and more preferably a hydrogen atom.

Each m in Formula (1) independently denotes an integer of 1 to 5, preferably 1, 2, or 3, more preferably 1 or 2, and yet more preferably 1.

Each n in Formula (1) independently denotes an integer of 1 or more, and preferably 1, 2, or 3. It is preferable that all four n are the same, and it is more preferable that all n are 1.

Each p in Formula (1) independently denotes an integer of 0 to 4, preferably an integer of 0 to 2, more preferably 0 or 1, and yet more preferably 0.

Each q in Formula (1) independently denotes an integer of 0 to 4, preferably an integer of 0 to 2, more preferably 0 or 1, and yet more preferably 0.

X in Formula (1) denotes a tetravalent organic group having 1 to 9 carbons.

The tetravalent organic group having 1 to 9 carbons is not particularly limited, and is preferably a carbon atom or a tetravalent hydrocarbon group, and more preferably a carbon atom, a non-ring structure-containing tetravalent alkanetetrayl group, or a tetravalent arenetetrayl group.

Specific preferred examples of X include a carbon atom, an ethanetetrayl group, a propanetetrayl group, a butanetetrayl group, a pentanetetrayl group, a hexanetetrayl group, a cyclohexanetetrayl group, and a benzenetetrayl group.

Furthermore, the four bonding positions in the tetravalent organic group may be any position. For example, in the case of an ethanetetrayl group, it may be either a 1,1,2,2-ethanetetrayl group or a 1,1,1,2-ethanetetrayl group. The benzenetetrayl group may be any of a 1,2,3,4-benzenetetrayl group, a 1,2,3,5-benzenetetrayl group, and a 1,2,4,5-benzenetetrayl group.

Among them, X is more preferably a carbon atom that can have a regular tetrahedral structure.

Moreover, in Formula (1), all of the four groups bonding to X are preferably the same groups.

With regard to the compound represented by Formula (1), one type thereof may be used on its own or two or more types of compounds may be used in combination.

With regard to the molecular weight of the compound represented by Formula (1), the weight-average molecular weight (Mw) is preferably at least 300. When it is at least 300, since the volatility is low, the concentration thereof in a film does not decrease, and an intended function is sufficiently exhibited.

A method for synthesizing the compound represented by Formula (1) is not particularly limited, and it may be synthesized by a known method or by application thereof. For example, an esterification reaction, an etherification reaction, a Sonogashira coupling reaction, etc. can be cited.

Specific examples of the compound represented by Formula (1) that can be used in the present invention are listed below, but the present invention is not limited thereto.

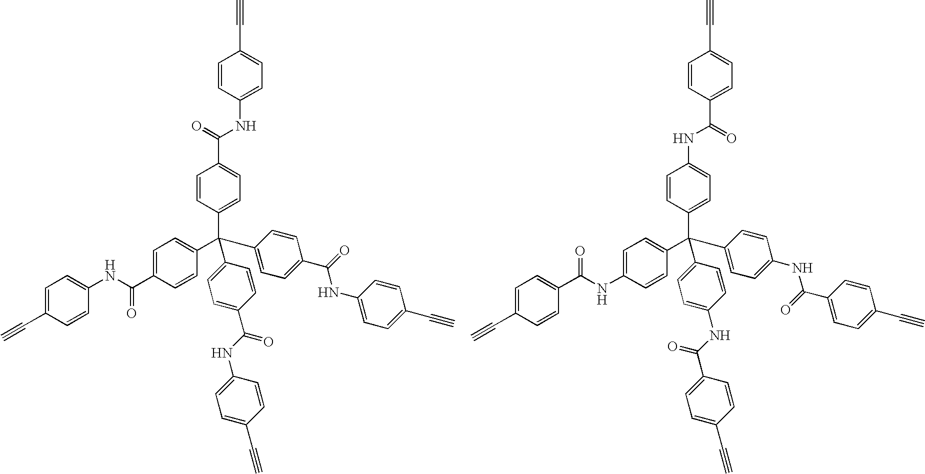

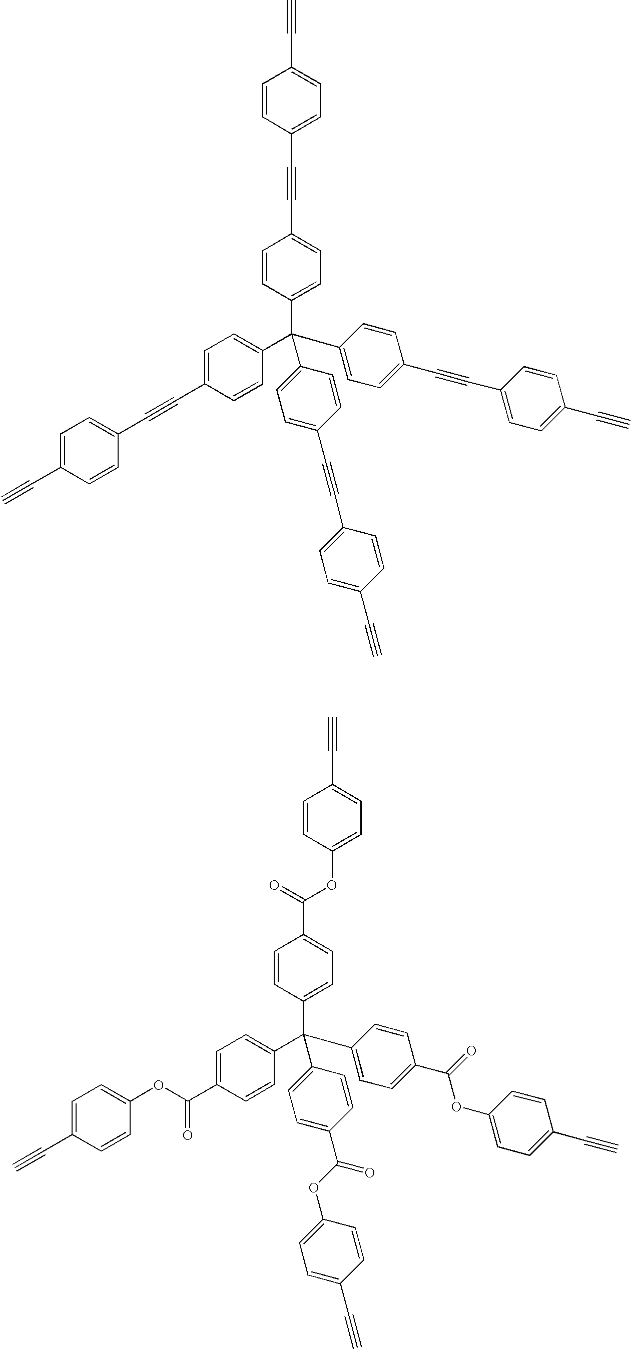

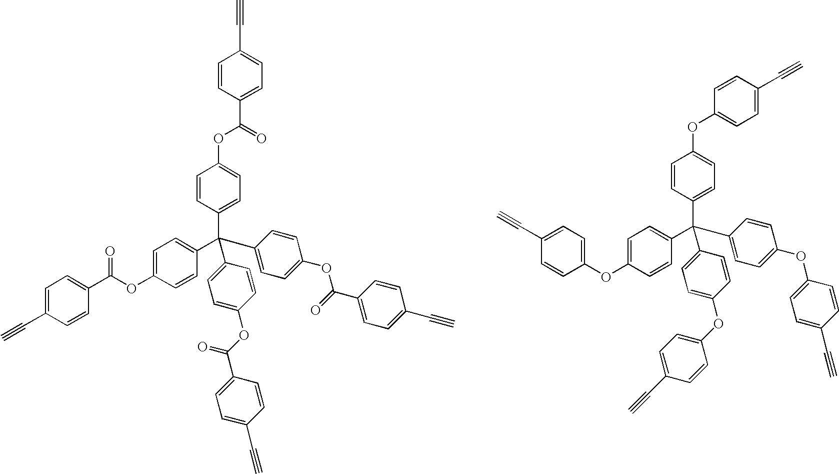

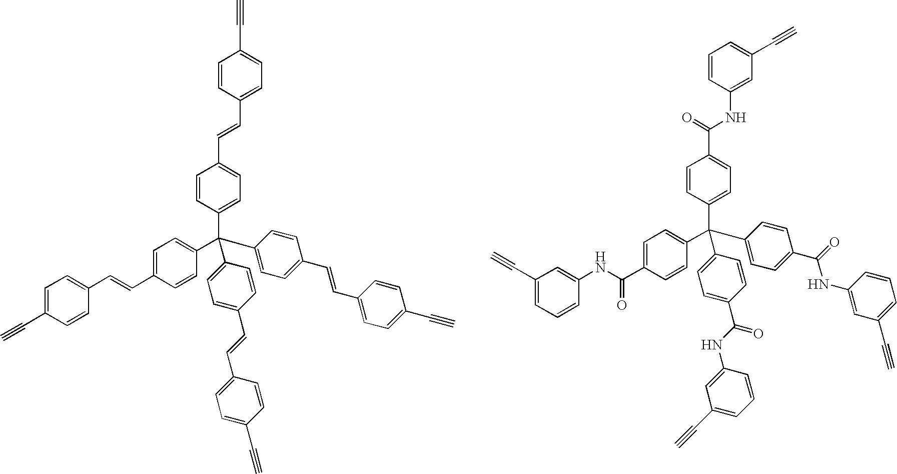

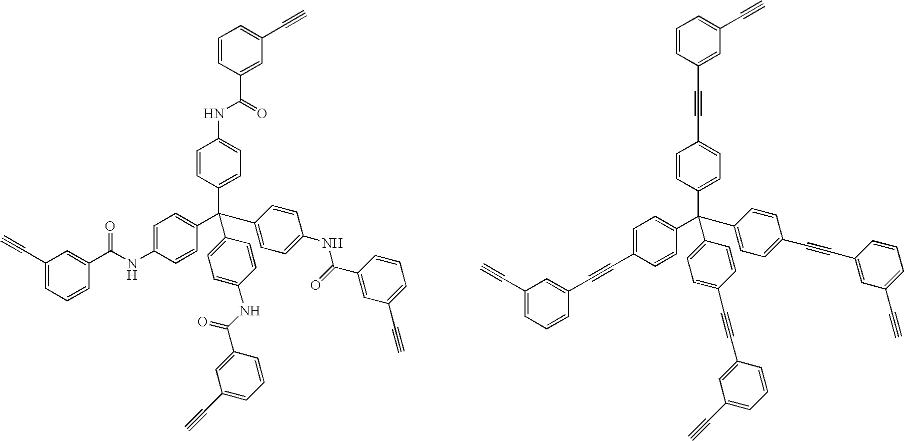

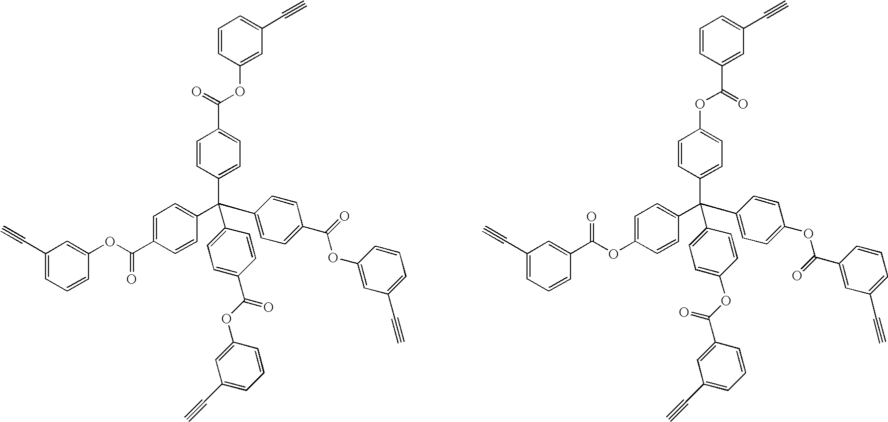

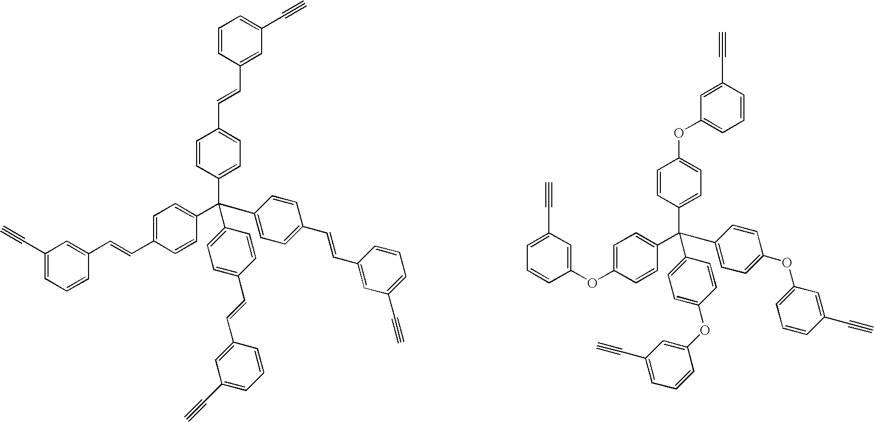

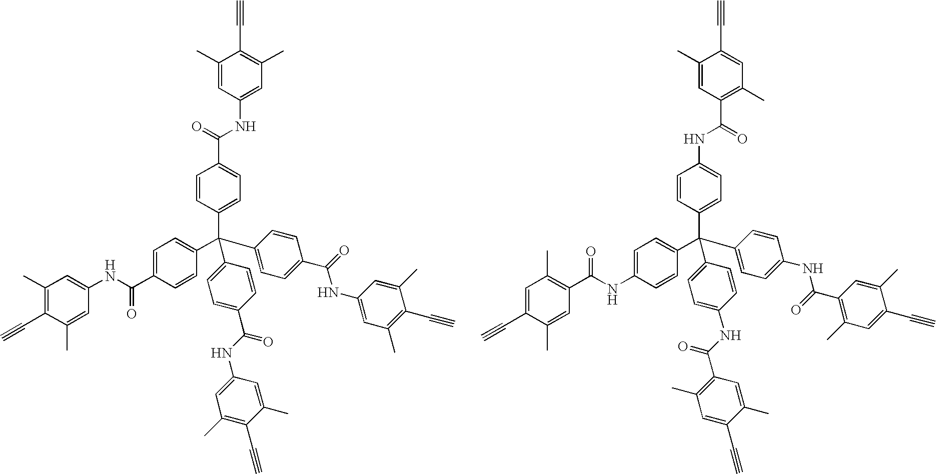

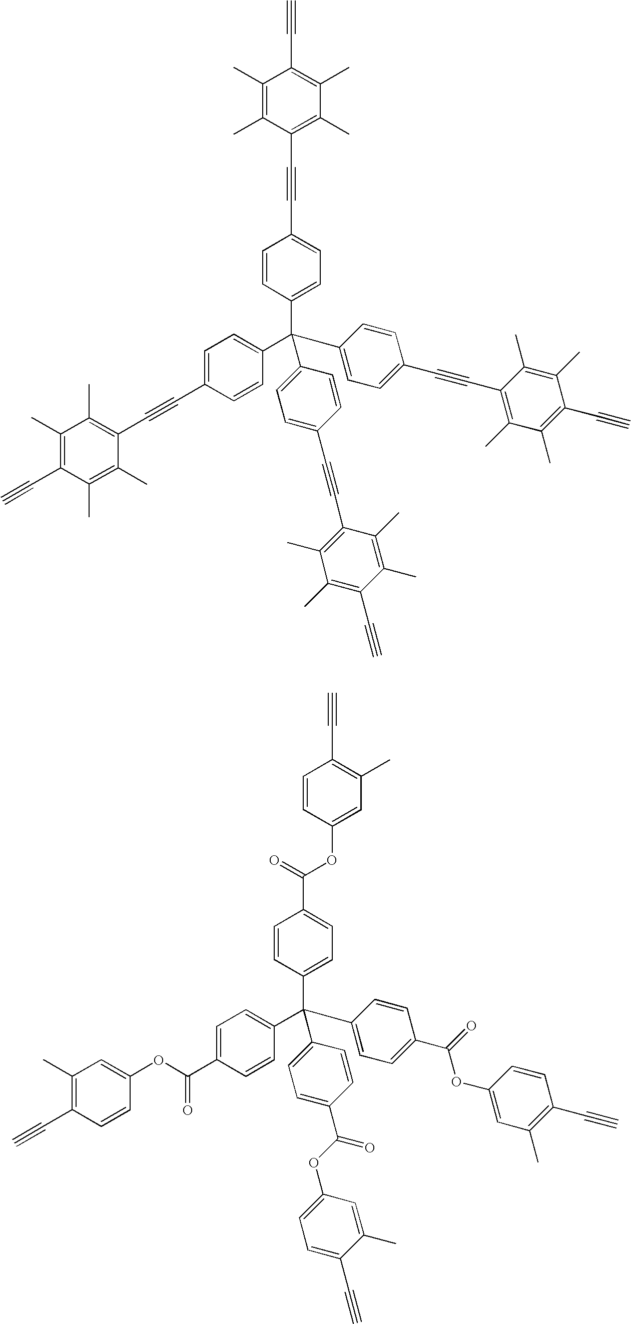

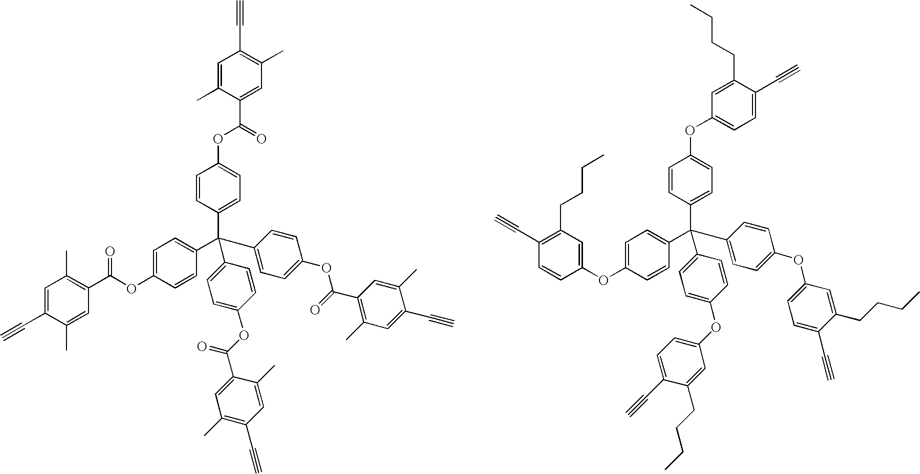

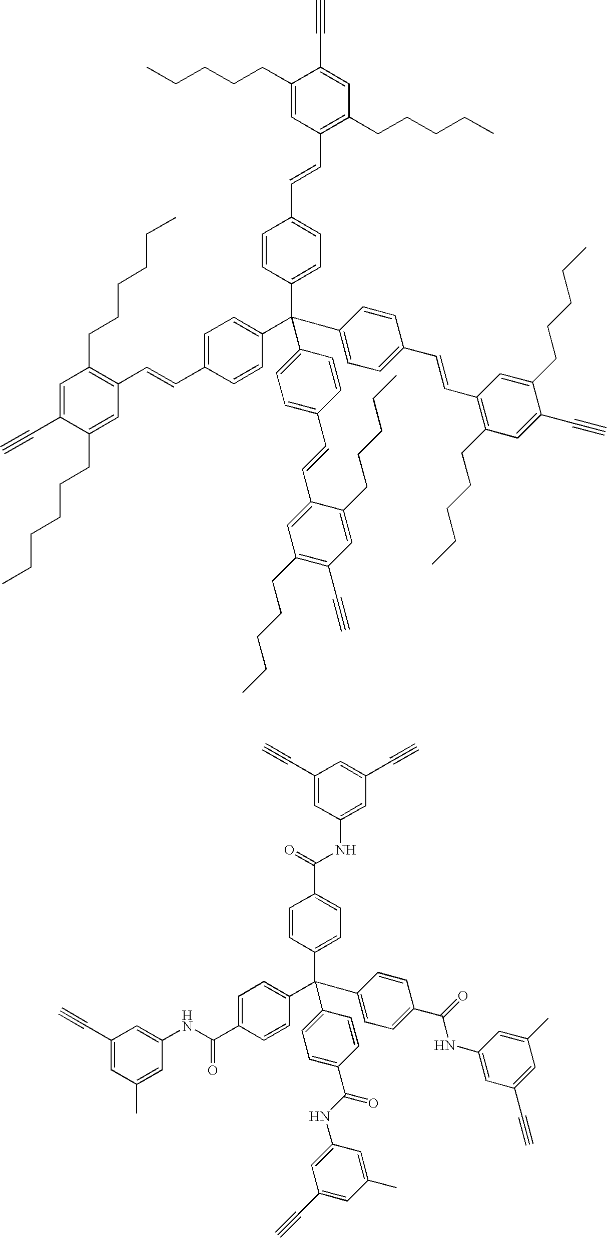

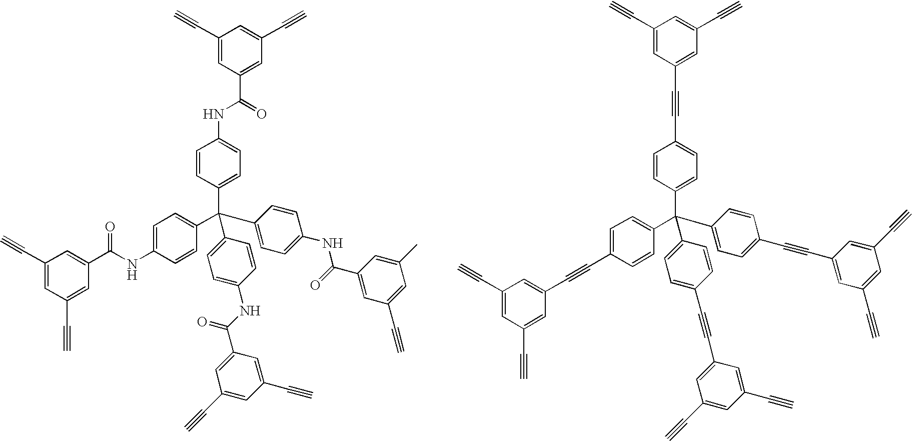

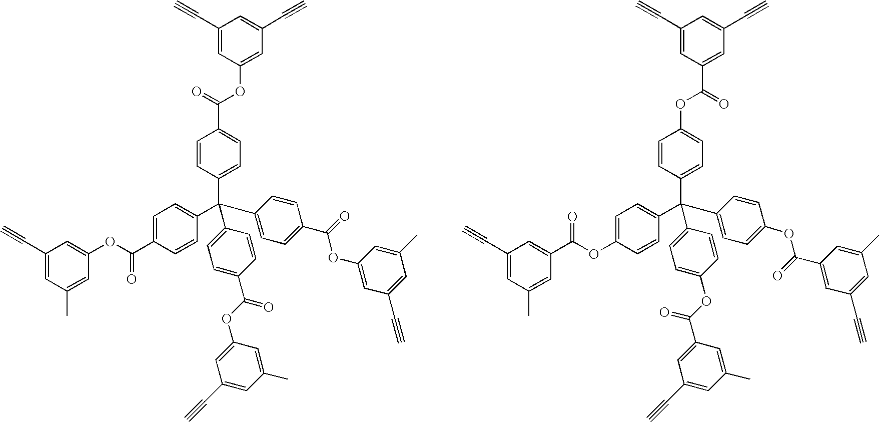

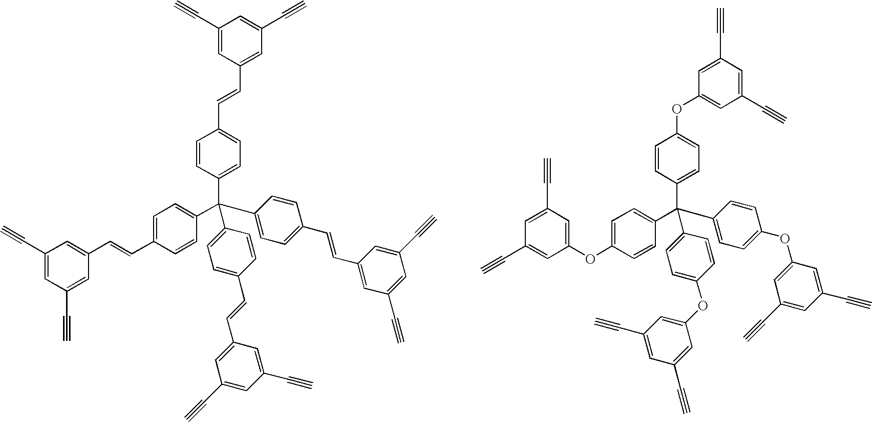

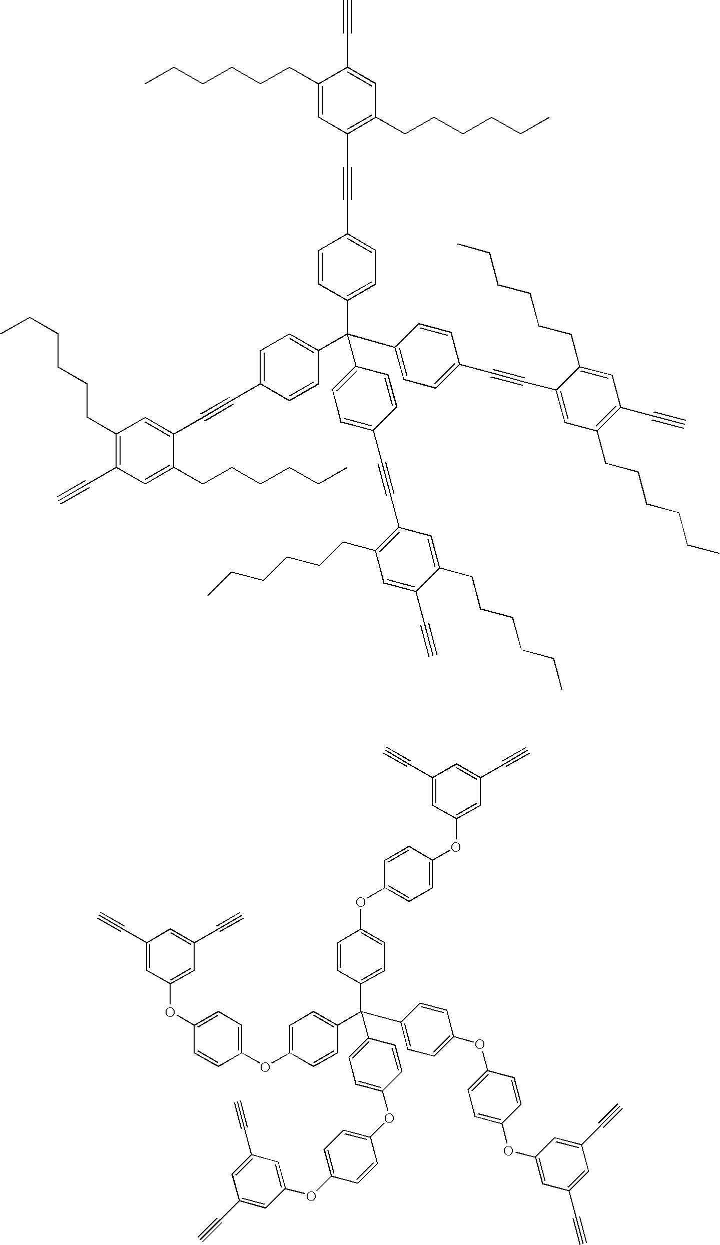

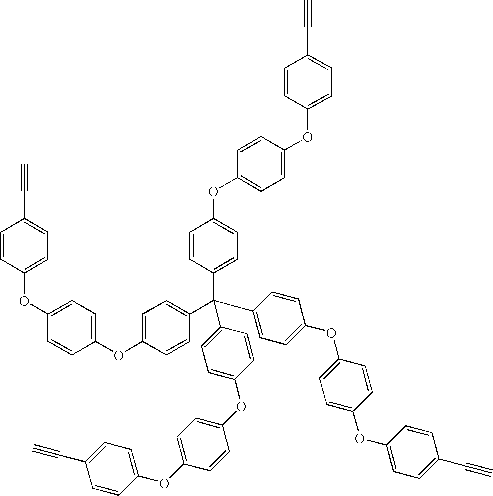

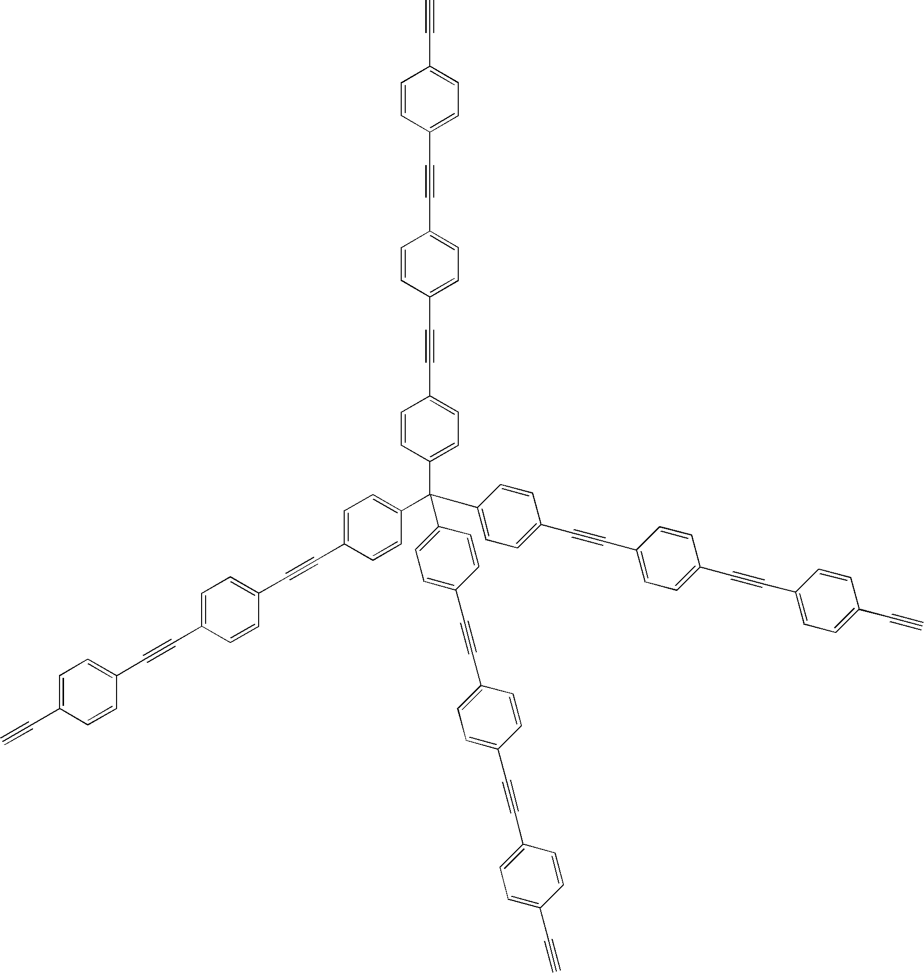

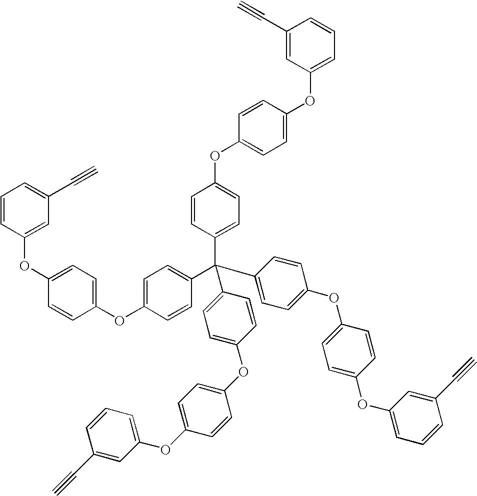

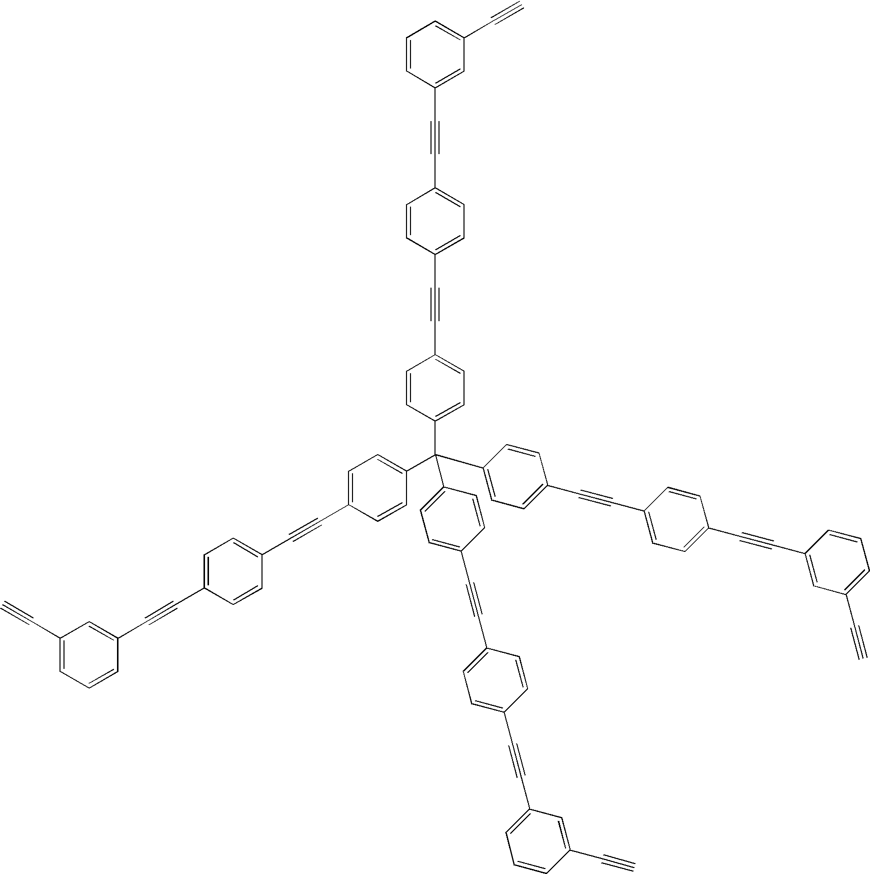

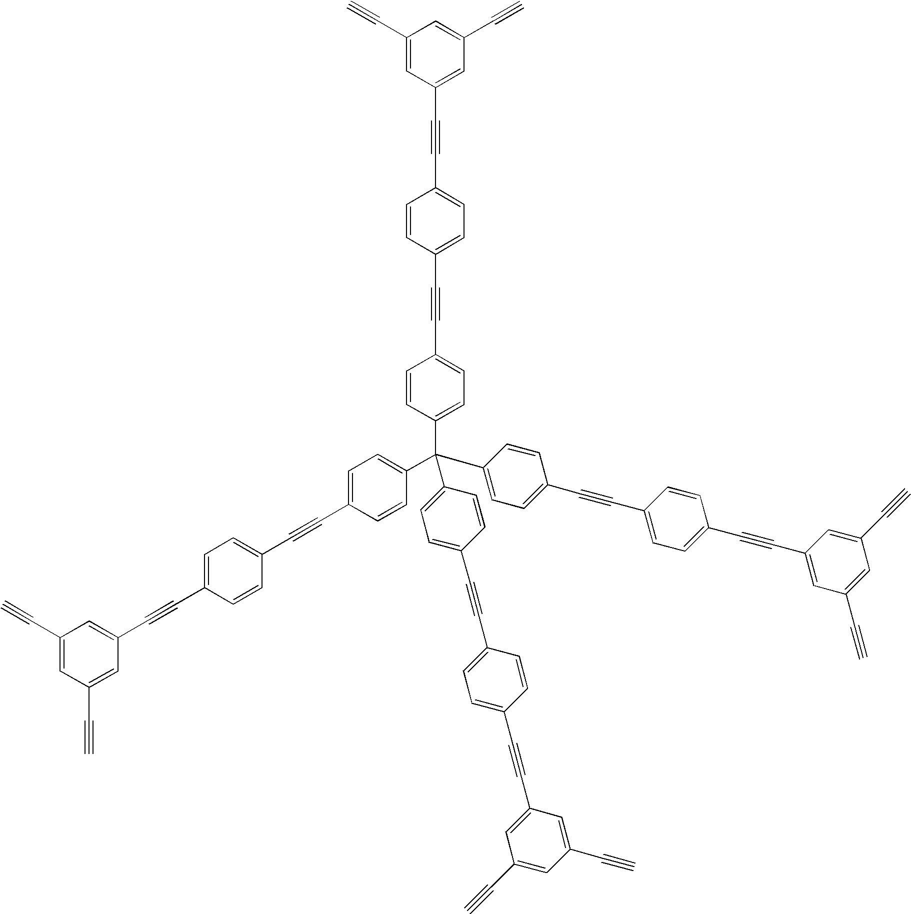

Among them, it is preferable to use a compound selected from the group consisting of compound (a) to compound (h) below, it is more preferable to use a compound selected from the group consisting of compound (a) to compound (d), compound (g), and compound (h), and it is yet more preferable to use a compound selected from the group consisting of compound (a), compound (b), and compound (g).

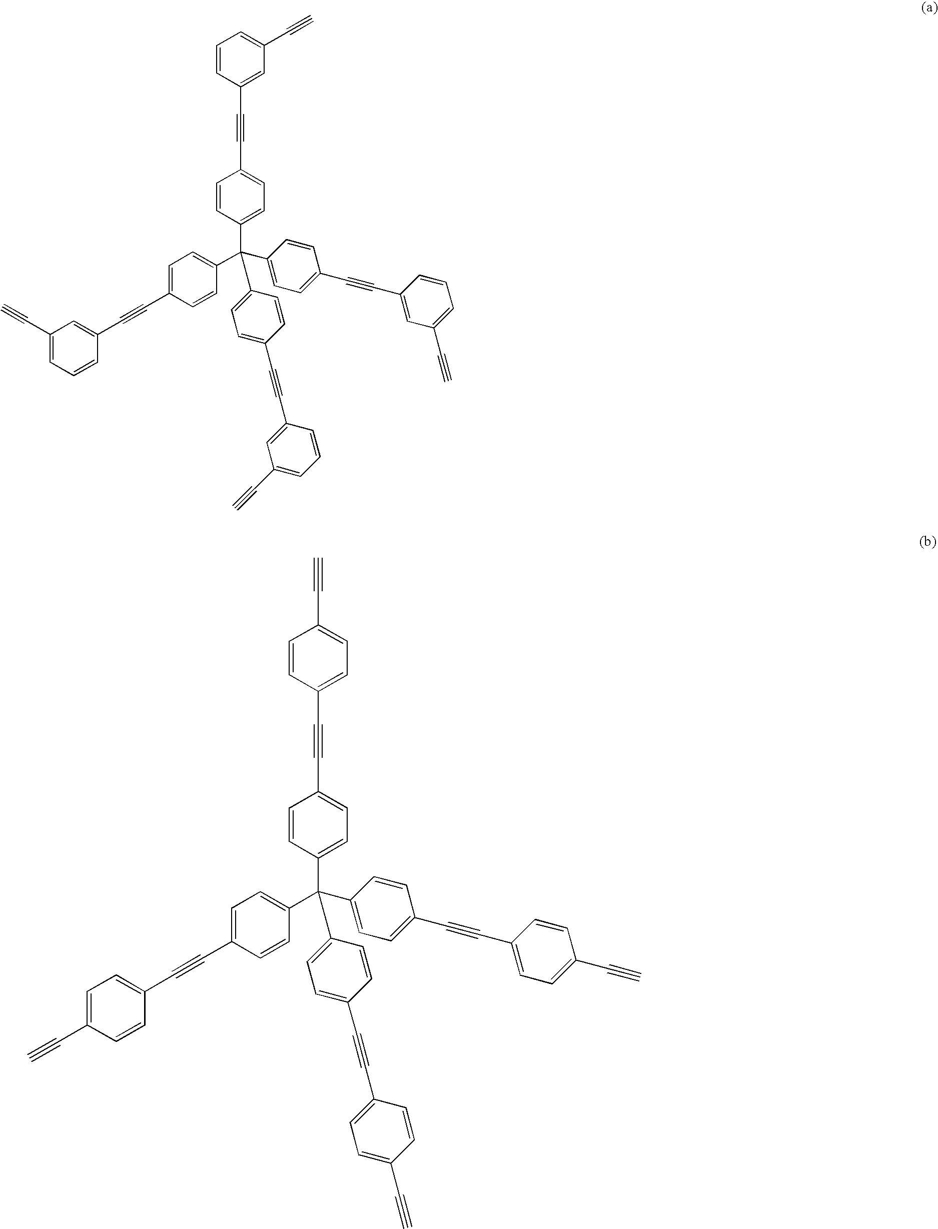

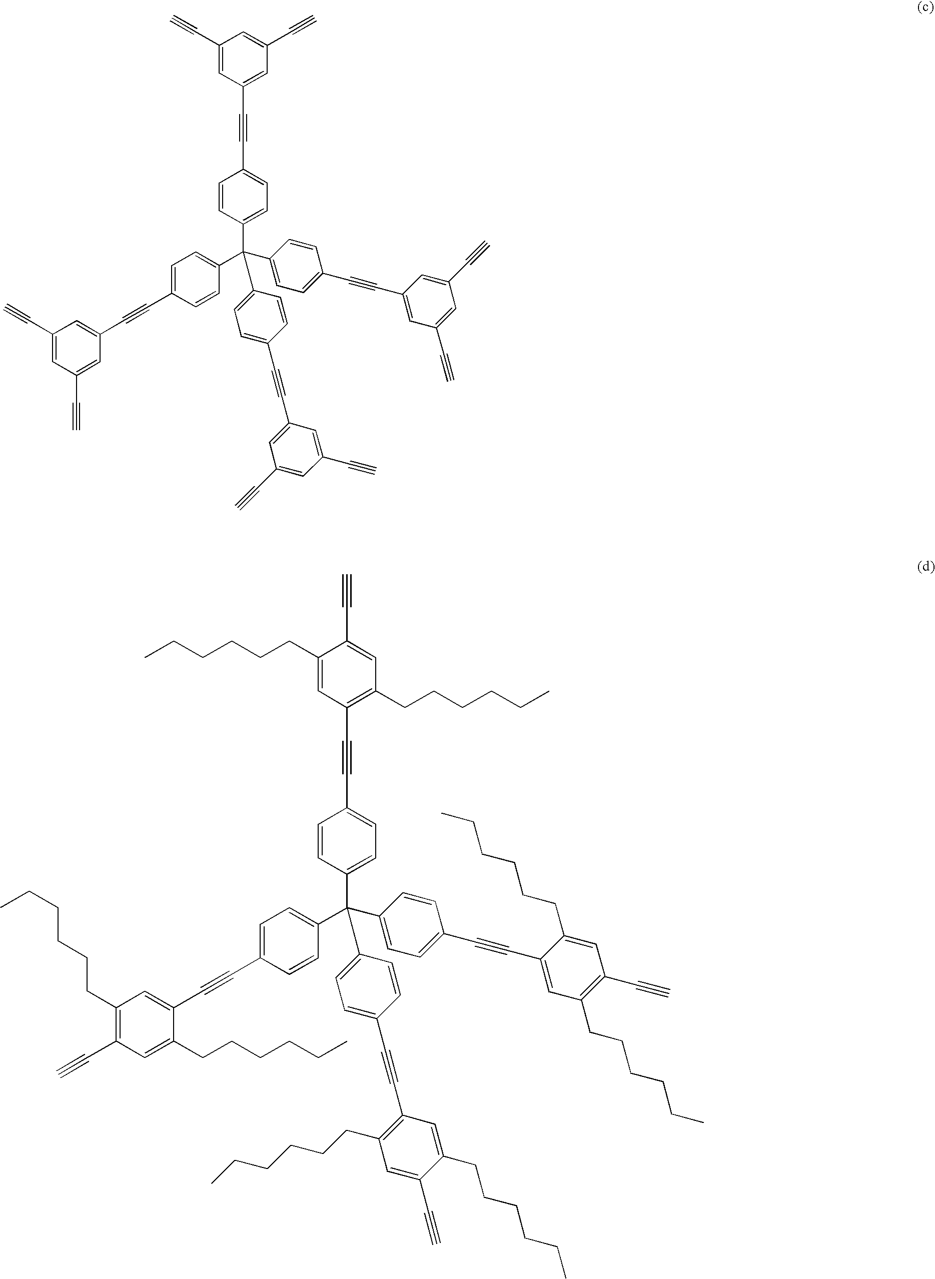

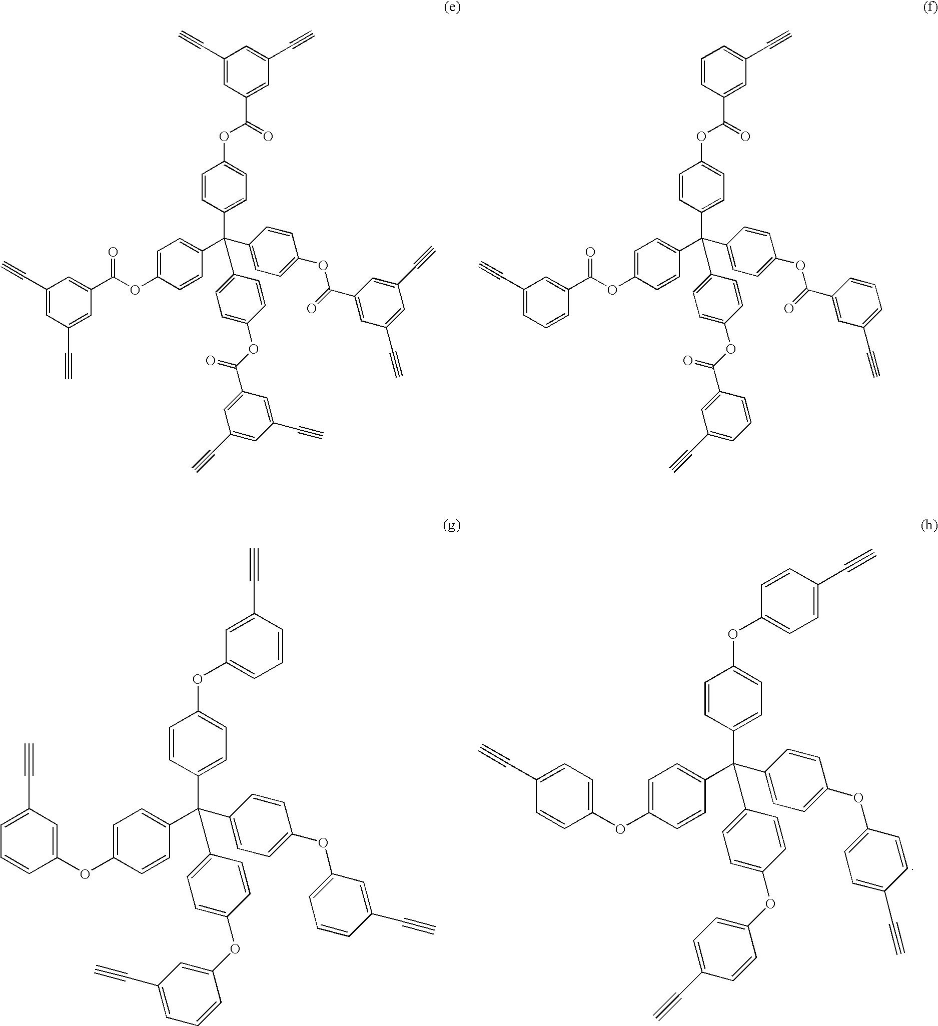

The amount of compound represented by Formula (1) added in the film forming composition of the present invention is preferably 0.1 to 80 wt % relative to the solids content of the film forming composition, more preferably 1 to 70 wt %, and particularly preferably 5 to 50 wt %. The solids content referred to here corresponds to all the components constituting a film obtained using the composition.

### Polymer Polymerized Using at Least Compound Represented by Formula (1)

The film forming composition of the present invention comprises a compound represented by Formula (1) and/or a polymer polymerized using at least a compound represented by Formula (1), and preferably comprises a polymer polymerized using at least a compound represented by Formula (1).

The polymer polymerized using at least a compound represented by Formula (1) may be a polymer polymerized using one type or two or more types of compounds represented by Formula (1) alone, or a copolymer polymerized using one type or two or more types of compounds represented by Formula (1) and one type or two or more types of other polymerizable compounds, and is preferably a polymer polymerized using a compound represented by Formula (1) alone.

Preferred examples of the compound represented by Formula (1) used in the polymer are the same as those described above as preferred compounds for the compound represented by Formula (1).

With regard to the compound represented by Formula (1) used in a starting material of the polymer, one type thereof may be used on its own, or two or more types may be used in combination.

Said other polymerizable compound is not particularly limited as long as it is copolymerizable with a compound represented by Formula (1), and a known polymerizable compound may be used.

Furthermore, when said other polymerizable compound is used, one type of said other polymerizable compound may be used on its own or two or more types thereof may be used in combination.

The weight-average molecular weight of the polymer polymerized using at least a compound represented by Formula (1) above is preferably 1,000 to 500,000, more preferably 5,000 to 200,000, and particularly preferably 10,000 to 100,000.

A process for producing the polymer polymerized using at least a compound represented by Formula (1) is not particularly limited, but a process comprising a step of carrying out polymerization using at least a compound represented by Formula (1) by heating only or in the presence of a non-metallic polymerization initiator is preferable.

A polymerization reaction of a compound represented by Formula (1) takes place due to the carbon-carbon triple bond. A polymerization reaction of a compound represented by Formula (1) is preferably carried out by heating only or in the presence of a non-metallic polymerization initiator. For example, a monomer having a carbon-carbon triple bond may be polymerized by heating only, or in the presence of a polymerization initiator that exhibits activity by generating a free radical such as a carbon radical or an oxygen radical upon heating.

As the polymerization initiator, an organic peroxide or an organic azo compound is preferably used, and an organic peroxide is particularly preferable.

As the organic peroxide, a ketone peroxide such as PERHEXA H, a peroxyketal such as PERHEXA TMH, a hydroperoxide such as PERBUTYL H-69, a dialkyl peroxide such as PERCUMYL D, PERBUTYL C, or PERBUTYL D, a diacyl peroxide such as NYPER BW, a peroxyester such as PERBUTYL Z or PERBUTYL L, or a peroxydicarbonate such as PEROYL TCP, which are commercially available from NOF Corporation, is preferably used.

As the organic azo compound, an azonitrile compound such as V-30, V-40, V-59, V-60, V-65, or V-70, an azoamide compound such as VA-080, VA-085, VA-086, VF-096, VAm-110, or VAm-111, a cyclic azoamidine compound such as VA-044 or VA-061, or an azoamidine compound such as V-50 or VA-057, which are commercially available from Wako Pure Chemical Industries, Ltd., is preferably used.

With regard to the polymerization initiator, only one type thereof may be used, or two or more types may be used as a mixture.

The amount of polymerization initiator used, per mole of the compound represented by Formula (1), is preferably 0.001 to 2 moles, more preferably 0.01 to 1 moles, and particularly preferably 0.05 to 0.5 moles.

The polymerization reaction of the compound represented by Formula (1) may be carried out in the presence of a transition metal catalyst. For example, a monomer having a polymerizable carbon-carbon double bond or carbon-carbon triple bond is preferably polymerized using a Pd-based catalyst such as tetrakistriphenylphosphine palladium (Pd(PPh₃)₄) or palladium acetate (Pd(OAc)₂), a Ziegler-Natta catalyst, a Ni-based catalyst such as nickel acetylacetonate, a W-based catalyst such as WCl₆, an Mo-based catalyst such as MoCl₅, a Ta-based catalyst such as TaCl₅, an Nb-based catalyst such as NbCl₅, a Rh-based catalyst, a Pt-based catalyst, etc.

With regard to the transition metal catalyst, only one type thereof may be used, or two or more types may be used as a mixture.

The amount of transition metal catalyst used, per mole of the compound represented by Formula (1), is preferably 0.001 to 2 moles, more preferably 0.01 to 1 moles, and particularly preferably 0.05 to 0.5 moles.

The amount added of the polymer polymerized using at least a compound represented by Formula (1) in the film forming composition of the present invention, relative to the solids content of the film forming composition, is preferably 1 to 100 wt %, more preferably 10 to 100 wt %, and particularly preferably 20 to 100 wt %. The solids content referred to here corresponds to all the components constituting a film obtained using the composition.

Compound having Cage Structure and Polymer having Cage Structure

The film forming composition of the present invention preferably comprises a compound having a cage structure and/or a polymer having a cage structure.

The ‘cage structure’ referred to in the present invention means a structure whose volume is defined by a plurality of rings formed from covalently bonded atoms, and in which a point within the volume cannot leave the volume without passing through a ring.

For example, the adamantane structure is considered to be a cage structure. In contrast thereto, a cyclic structure having a single bridge such as norbornane (bicyclo[2.2.1]heptane) is not considered to be a cage structure since the rings of a singly bridged cyclic compound do not define a volume.

The cage structure may comprise either saturated or unsaturated bonds, and may comprise a hetero atom such as oxygen, nitrogen, or sulfur; it is preferably a cage structure formed from a hydrocarbon, and from the viewpoint of low permittivity is more preferably a cage structure formed from a saturated hydrocarbon.

The cage structure in the present invention is preferably an adamantane structure, a biadamantane structure, a diamantane structure, a bi(diamantane) structure, a triamantane structure, a tetramantane structure, or a dodecahedrane structure, more preferably an adamantane structure, a biadamantane structure, a diamantane structure, a triamantane structure, or a tetramantane structure, yet more preferably an adamantane structure, a biadamantane structure, or a diamantane structure and, from the viewpoint of low permittivity, particularly preferably a biadamantane structure or a diamantane structure.

The above-mentioned structures are shown below. The dodecahedrane structure is a regular dodecahedron hydrocarbon structure in which the 20 vertices of the regular dodecahedron are each a carbon atom. Furthermore, the portion connecting two adamantane structures in the biadamantane structure may be at any position, but they are preferably connected via bridgehead positions thereof. Similarly, with regard to the bi(diamantane) structure, the portion connecting two diamantane structures may be at any position, but they are preferably connected via bridgehead positions thereof.

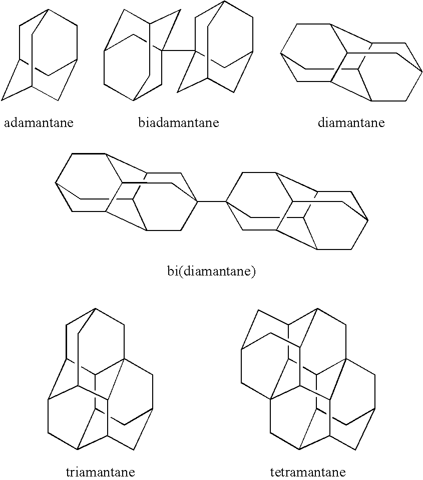

The cage structure may have one or more substituents.

Examples of the substituent include a halogen atom (a fluorine atom, a chlorine atom, a bromine atom, or an iodine atom), a straight chain, branched, or cyclic alkyl group having 1 to 10 carbons (methyl, t-butyl, cyclopentyl, cyclohexyl, etc.), an alkenyl group having 2 to 10 carbons (vinyl, propenyl, etc.), an alkynyl group having 2 to 10 carbons (ethynyl, phenylethynyl, etc.), an aryl group having 6 to 20 carbons (phenyl, 1-naphthyl, 2-naphthyl, etc.), an acyl group having 2 to 10 carbons (benzoyl, etc.), an alkoxycarbonyl group having 2 to 10 carbons (methoxycarbonyl, etc.), a carbamoyl group having 1 to 10 carbons (N,N-diethylcarbamoyl, etc.), an aryloxy group having 6 to 20 carbons (phenoxy, etc.), an arylsulfonyl group having 6 to 20 carbons (phenylsulfonyl, etc.), a nitro group, a cyano group, and a silyl group (triethoxysilyl, methyldiethoxysilyl, trivinylsilyl, etc.).

The cage structure is preferably a 2- to 4-valent group. In this case, the group bonded to the cage structure may be a monovalent substituent or a di- or higher-valent linking group. The cage structure is preferably a di- or tri-valent group, and particularly preferably a divalent group.

The film forming composition of the present invention preferably comprises a polymer having a cage structure, and more preferably a polymer of a monomer having a cage structure.

The monomer referred to here means one that polymerizes to form a dimer or higher polymer. This polymer may be a homopolymer or a copolymer.

A polymerization reaction of the monomer proceeds via a polymerizable group with which the monomer is substituted. The polymerizable group referred to here means a reactive substituent that allows the monomer to polymerize. This polymerization reaction may be any polymerization reaction, and examples thereof include radical polymerization, cationic polymerization, anionic polymerization, ring-opening polymerization, polycondensation, polyaddition, addition condensation, and transition metal catalyzed polymerization.

The polymerization reaction of the monomer is preferably carried out in the presence of a nonmetallic polymerization initiator. For example, a monomer having a polymerizable carbon-carbon double bond or carbon-carbon triple bond may be polymerized in the presence of a polymerization initiator that generates a free radical such as a carbon radical or an oxygen radical upon heating to thus exhibit activity.

As the radical polymerization initiator, an organic peroxide or an organic azo compound is preferably used, and an organic peroxide is particularly preferable. As the organic peroxide and the organic azo compound, those described above may be used preferably.

With regard to the polymerization initiator, only one type thereof may be used or two or more types thereof may be used as a mixture.

The amount of polymerization initiator used, per mole of monomer, is preferably 0.001 to 2 moles, more preferably 0.01 to 1 moles, and particularly preferably 0.05 to 0.5 moles.

It is also preferable to carry out a monomer polymerization reaction in the presence of a transition metal catalyst. As the transition metal catalyst, those described above may preferably be used.

With regard to the transition metal catalyst, only one type thereof may be used or two or more types thereof may be used as a mixture.

The amount of transition metal catalyst used, per mole of monomer, is preferably 0.001 to 2 moles, more preferably 0.01 to 1 moles, and particularly preferably 0.05 to 0.5 moles.

The process for producing the polymer having a cage structure is not particularly limited but is preferably a process comprising a step of carrying out polymerization using a monomer having a cage structure in the presence of a polymerization initiator, and is more preferably a process comprising a step of carrying out polymerization using a monomer having a cage structure in the presence of a radical initiator or a transition metal catalyst.

The cage structure in the present invention may be substituted as a pendant group in the polymer or may be part of the polymer main chain, but a configuration in which it is part of the polymer main chain is preferable.

The configuration in which it is part of the polymer main chain means that if the cage compound is removed from the polymer the polymer chain is cleaved. In this configuration, the cage structure is either directly bonded via single bonds or bonded via appropriate divalent linking groups. Examples of the linking group include —C(R¹¹)(R¹²)—, —C(R¹³)═C(R¹⁴)—, —C≡C—, an arylene group, —CO—, —O—, —SO₂—, —N(R¹⁵)—, —Si(R¹⁶)(R¹⁷)—, and a group in which they are combined. Here, R¹¹ to R¹⁷ independently denote a hydrogen atom, an alkyl group, an alkenyl group, an alkynyl group, or an aryl group. These linking groups may have a substituent, and preferred examples of the substituent include the above-mentioned substituents.

Among them, more preferred linking groups are —C(R¹¹)(R¹²)—, —CH═CH—, —C≡C—, an arylene group, —O—, —Si(R¹⁶)(R¹⁷)—, and a group in which they are combined, and from the viewpoint of low permittivity —C(R¹¹)(R¹²)— and —CH═CH— are particularly preferable.

The weight-average molecular weight of the polymer having a cage structure is preferably 1,000 to 500,000, more preferably 5,000 to 200,000, and particularly preferably 10,000 to 100,000.

Furthermore, the polymer having a cage structure may be contained in the film forming composition of the present invention as a resin composition having a molecular weight distribution.

The molecular weight of the compound having a cage structure is preferably 150 to 3,000, more preferably 200 to 2,000, and particularly preferably 220 to 1,000.

The polymer having a cage structure that can be used in the present invention is preferably a polymer of a monomer having a polymerizable carbon-carbon double bond or carbon-carbon triple bond, and is more preferably a polymer of a compound represented by Formulae (2) to (7) below.

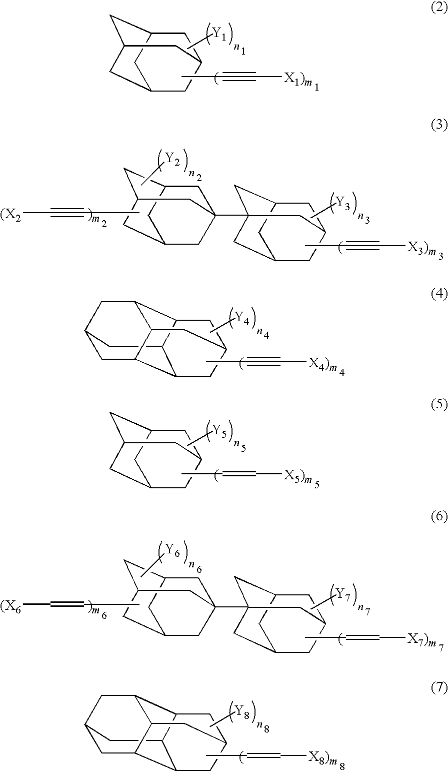

(In Formulae (2) to (7), X₁ to X₈ independently denote a hydrogen atom, an alkyl group, an alkenyl group, an alkynyl group, an aryl group, a silyl group, an acyl group, an alkoxycarbonyl group, a carbamoyl group, etc., Y₁ to Y₈ independently denote a halogen atom, an alkyl group, an aryl group, or a silyl group, m₁ and m₅ denote an integer of 1 to 16, n₁ and n₅ denote an integer of 0 to 15, m₂, m₃, m₆, and m₇ independently denote an integer of 1 to 15, n₂, n₃, n₆, and n₇ denote an integer of 0 to 14, m₄ and m₈ denote an integer of 1 to 20, and n₄ and n₈ denote an integer of 0 to 19.)

In Formulae (2) to (7), X₁ to X₈ independently denote a hydrogen atom, an alkyl group having 1 to 10 carbons, an alkenyl group having 2 to 10 carbons, an alkynyl group having 2 to 10 carbons, an aryl group having 6 to 20 carbons, a silyl group having 0 to 20 carbons, an acyl group having 2 to 10 carbons, an alkoxycarbonyl group having 2 to 10 carbons, a carbamoyl group having 1 to 20 carbons, etc. Among them, they are preferably a hydrogen atom, an alkyl group having 1 to 10 carbons, an aryl group having 6 to 20 carbons, a silyl group having 0 to 20 carbons, an acyl group having 2 to 10 carbons, an alkoxycarbonyl group having 2 to 10 carbons, or a carbamoyl group having 1 to 20 carbons, more preferably a hydrogen atom or an aryl group having 6 to 20 carbons, and particularly preferably a hydrogen atom.

In Formulae (2) to (7), Y₁ to Y₈ independently denote a halogen atom (fluorine, chlorine, bromine, etc.), an alkyl group having 1 to 10 carbons, an aryl group having 6 to 20 carbons, or a silyl group having 0 to 20 carbons, more preferably an optionally substituted alkyl group having 1 to 10 carbons or aryl group having 6 to 20 carbons, and particularly preferably an alkyl group (a methyl group, etc.).

X₁ to X₈ and Y₁ to Y₈ may be further substituted with another substituent.

In Formula (2) or Formula (5), m₁ and m₅ independently denote an integer of 1 to 16, preferably 1 to 4, more preferably 1 to 3, and particularly preferably 2.

In Formula (2) or Formula (5), n₁ and n₅ independently denote an integer of 0 to 15, preferably 0 to 4, more preferably 0 or 1, and particularly preferably 0.

In Formula (3) or Formula (6), m₂, m₃, m₆, and m₇ independently denote an integer of 1 to 15, preferably 1 to 4, more preferably 1 to 3, and particularly preferably 2.

In Formula (3) or Formula (6), n₂, n₃, n₆, and n₇ independently denote an integer of 0 to 14, preferably 0 to 4, more preferably 0 or 1, and particularly preferably 0.

In Formula (4) or Formula (7), m₄ and m₈ independently denote an integer of 1 to 20, preferably 1 to 4, more preferably 1 to 3, and particularly preferably 2.

In Formula (4) or Formula (7), n₄ and n₈ independently denote an integer of 0 to 19, preferably 0 to 4, more preferably 0 or 1, and particularly preferably 0.

The monomer having a cage structure that can be used in the present invention is preferably a compound represented by Formula (2), Formula (3), Formula (5), or Formula (6) above, more preferably a compound represented by Formula (2) or Formula (3), and particularly preferably a compound represented by Formula (3) above.

With regard to the compound having a cage structure and/or the polymer having a cage structure that can be used in the present invention, two or more types thereof may be used in combination, or two or more types of monomers having a cage structure that can be used in the present invention may be copolymerized.

The compound having a cage structure and the polymer having a cage structure preferably have sufficient solubility in an organic solvent. The solubility of the compound having a cage structure at 25° C. in cyclohexanone or anisole is preferably at least 3 wt %, more preferably at least 5 wt %, and particularly preferably at least 10 wt %.

Examples of the compound having a cage structure and the polymer having a cage structure that can be used in the present invention include polybenzoxazoles described in JP-A-11-322929, JP-A-2003-12802, and JP-A-2004-18593, a quinoline resin described in JP-A-2001-2899, polyaryl resins described in JP-PCT-2003-530464 (JP-PCT means a published Japanese translation of a PCT application), JP-PCT-2004-535497, JP-PCT-2004-504424, JP-PCT-2004-504455, JP-PCT-2005-501131, JP-PCT-2005-516382, JP-PCT-2005-514479, JP-PCT-2005-522528, JP-A-2000-100808, and U.S. Pat. No. 6509415, polyadamantanes described in JP-A-11-214382, JP-A-2001-332542, JP-A-2003-252982, JP-A-2003-292878, JP-A-2004-2787, JP-A-2004-67877, and JP-A-2004-59444, and polyimides described in JP-A-2003-252992 and JP-A-2004-26850.

Specific examples (M-1 to M-55) of the monomer having a cage structure that can be used in the present invention are listed below, but the present invention is not limited thereto. Et in the specific examples below denotes an ethyl group.

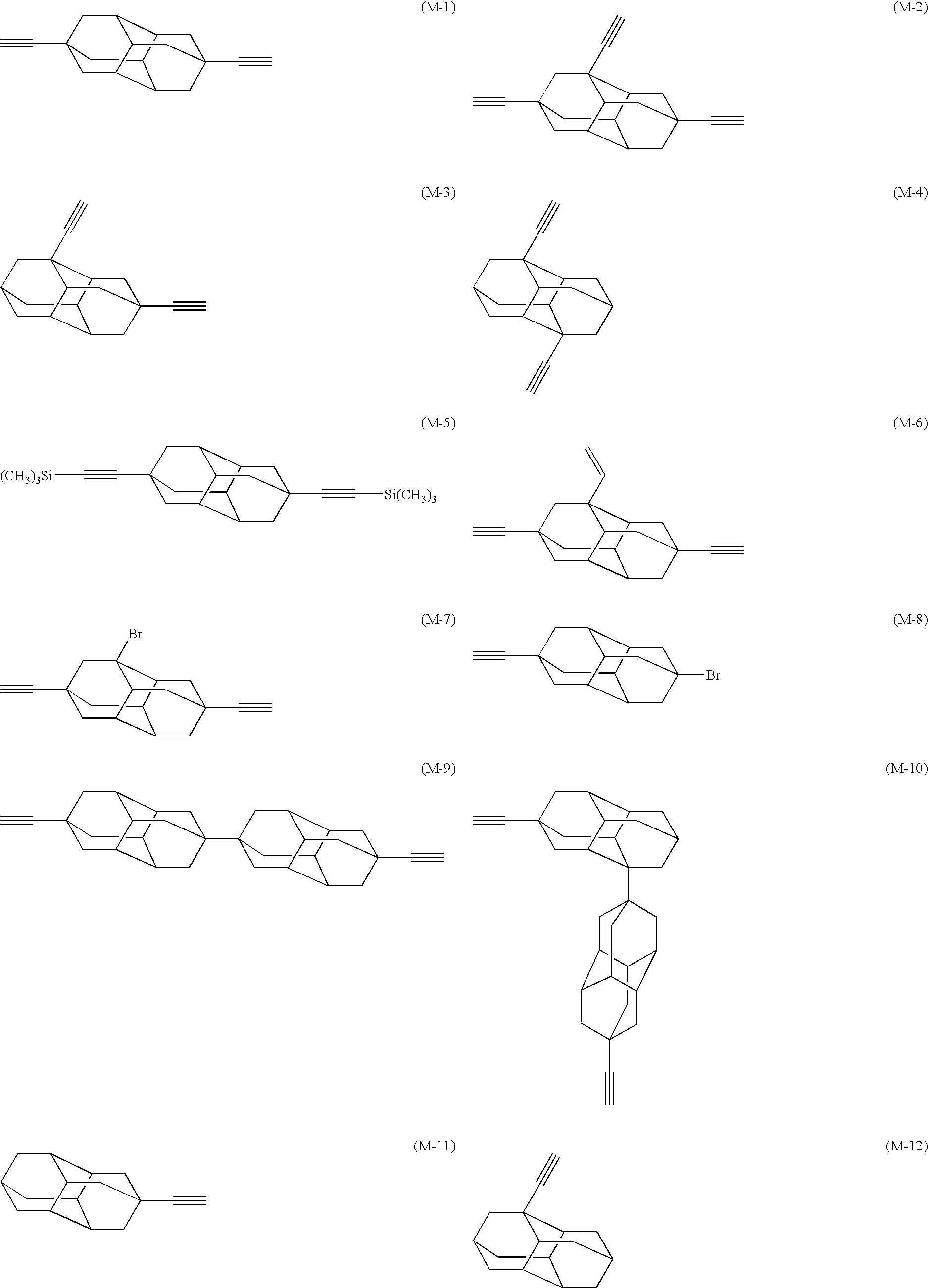

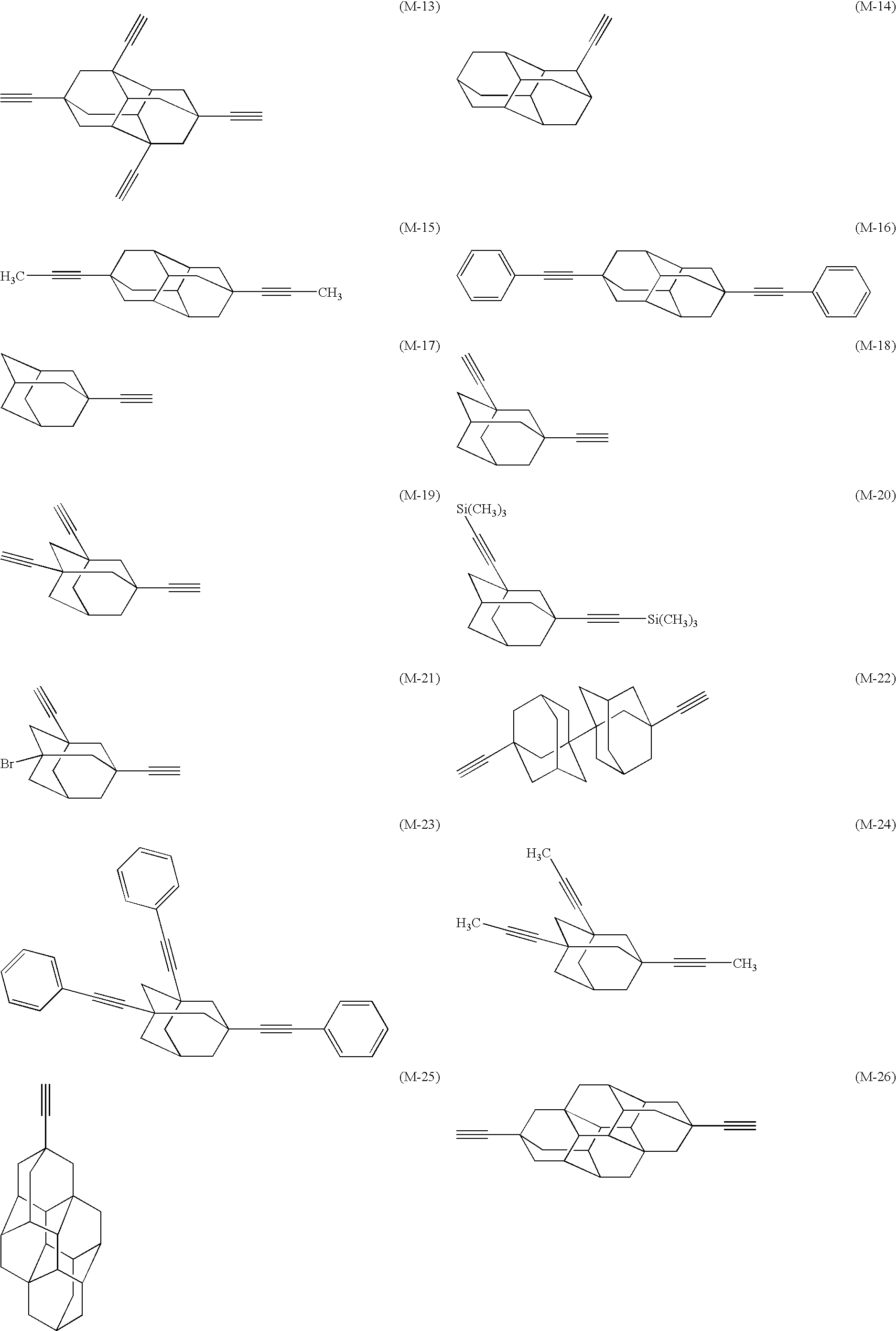

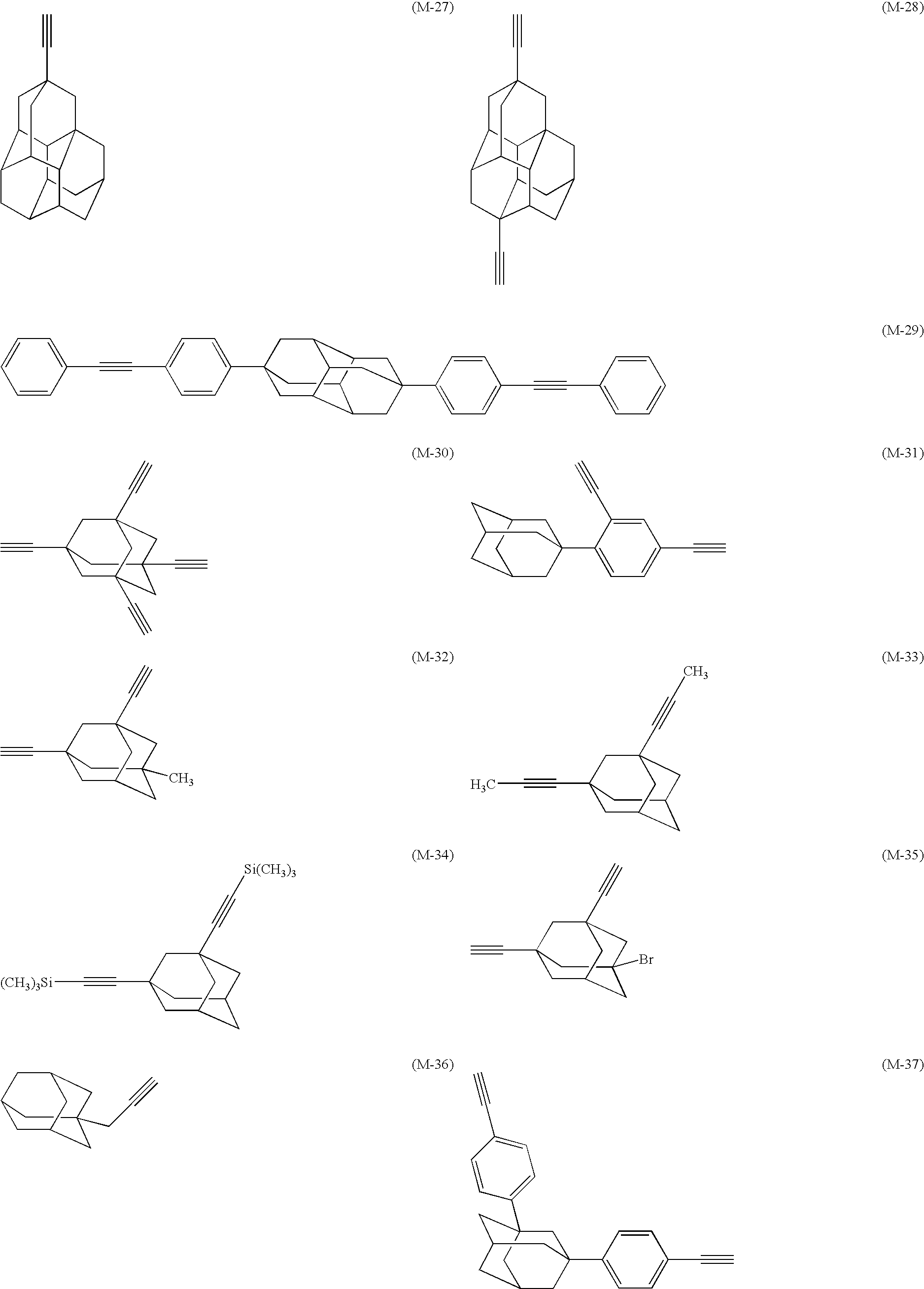

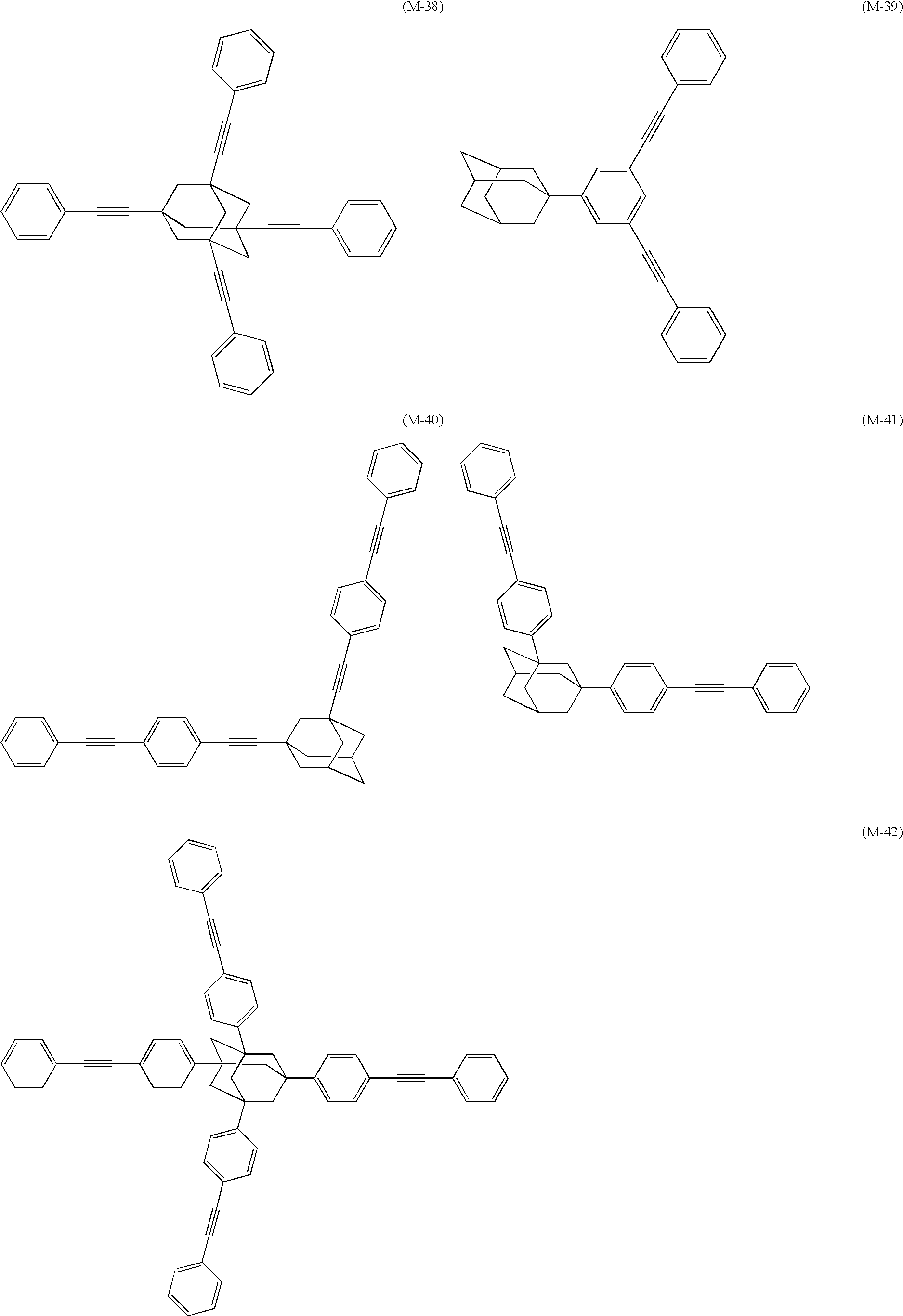

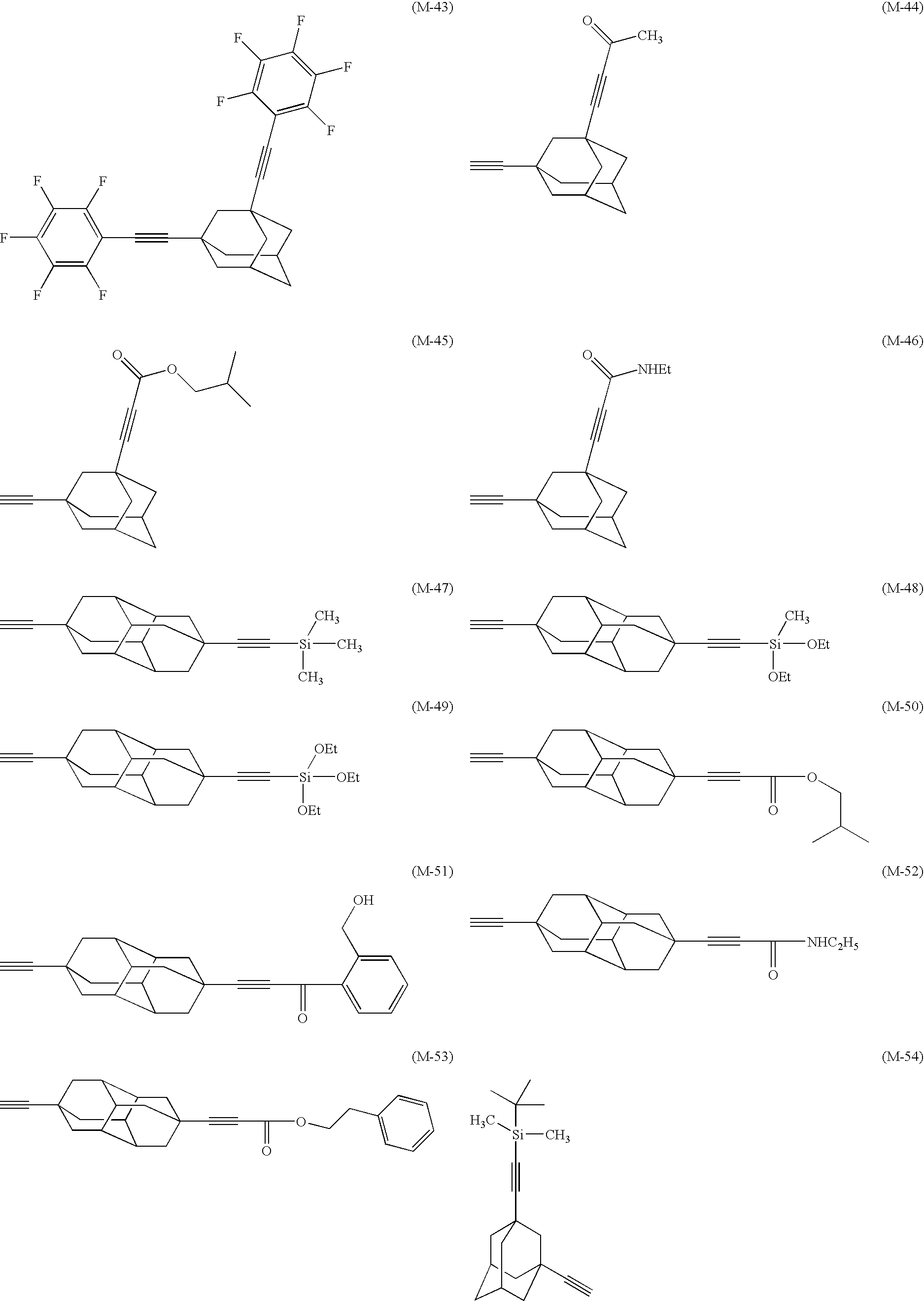

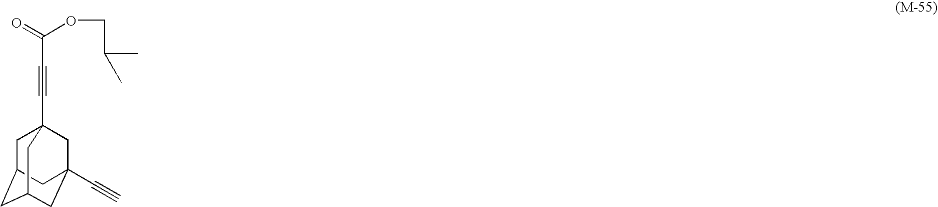

Furthermore, examples of the monomer having a cage structure that can be used in the present invention include those in which the —C≡C— in the specific examples above is replaced with —CH═CH—.

A solvent used in the polymerization reaction may be any solvent as long as a starting monomer is soluble therein at a required concentration and the properties of a film formed from the polymer obtained are not adversely affected. Examples thereof include water, alcohol-based solvents such as methanol, ethanol, and propanol, ketone-based solvents such as acetone, methyl ethyl ketone, methyl isobutyl ketone, cyclohexanone, and acetophenone, ester-based solvents such as ethyl acetate, butyl acetate, propylene glycol monomethyl ether acetate, γ-butyrolactone, and methyl benzoate, ether-based solvents such as dibutyl ether, anisole, and diphenyl ether, aromatic hydrocarbon-based solvents such as toluene, xylene, mesitylene, 1,2,4,5-tetramethylbenzene, pentamethylbenzene, isopropylbenzene, 1,4-diisopropylbenzene, t-butylbenzene, 1,4-di-t-butylbenzene, 1,3,5-triethylbenzene, 1,3,5-tri-t-butylbenzene, 4-t-butyl-orthoxylene, 1-methylnaphthalene, and 1,3,5-triisopropylbenzene, amide-based solvents such as N-methylpyrrolidinone and dimethylacetamide, halogen solvents such as carbon tetrachloride, dichloromethane, chloroform, 1,2-dichloroethane, chlorobenzene, 1,2-dichlorobenzene, and 1,2,4-trichlorobenzene, and aliphatic hydrocarbon-based solvents such as hexane, heptane, octane, and cyclohexane.

Among them, preferred solvents are acetone, methyl ethyl ketone, methyl isobutyl ketone, cyclohexanone, acetophenone, ethyl acetate, propylene glycol monomethyl ether acetate, γ-butyrolactone, anisole, diphenyl ether, tetrahydrofuran, toluene, xylene, mesitylene, 1,2,4,5-tetramethylbenzene, isopropylbenzene, t-butylbenzene, 1,4-di-t-butylbenzene, 1,3,5-tri-t-butylbenzene, 4-t-butyl-orthoxylene, 1-methylnaphthalene, 1,3,5-triisopropylbenzene, 1,2-dichloroethane, chlorobenzene, 1,2-dichlorobenzene, and 1,2,4-trichlorobenzene, more preferred solvents are tetrahydrofuran, γ-butyrolactone, anisole, toluene, xylene, mesitylene, isopropylbenzene, t-butylbenzene, 1,3,5-tri-t-butylbenzene, 1-methylnaphthalene, 1,3,5-triisopropylbenzene, 1,2-dichloroethane, chlorobenzene, 1,2-dichlorobenzene, and 1,2,4-trichlorobenzene, and particularly preferred solvents are γ-butyrolactone, anisole, diphenyl ether, mesitylene, t-butylbenzene, 1,3,5-triisopropylbenzene, 1,2-dichlorobenzene, and 1,2,4-trichlorobenzene. They may be used singly or as a mixture of two or more types thereof.

The concentration of the monomer in a reaction mixture is preferably 1 to 50 wt %, more preferably 5 to 30 wt %, and particularly preferably 10 to 20 wt %.

Optimum conditions for the polymerization reaction in the present invention depend on the type, concentration, etc. of polymerization initiator, monomer, and solvent, but the internal temperature is preferably 0° C. to 200° C., more preferably 50° C. to 170° C., and particularly preferably 100° C. to 150° C., and the time is preferably 1 to 50 hours, more preferably 2 to 20 hours, and particularly preferably 3 to 10 hours.

Furthermore, it is preferable to carry out the reaction under an inert gas atmosphere (e.g. nitrogen, argon, etc.) in order to suppress deactivation of the polymerization initiator by oxygen. The oxygen concentration during the reaction is preferably no greater than 100 ppm, more preferably no greater than 50 ppm, and particularly preferably no greater than 20 ppm.

The weight-average molecular weight of the polymer obtained by polymerization is preferably in the range of 1,000 to 500,000, more preferably 5,000 to 300,000, and particularly preferably 10,000 to 200,000.

Furthermore, the compound having a cage structure may be synthesized by, for example, reacting commercial diamantane as a starting material with bromine in the presence or absence of an aluminum bromide catalyst so as to introduce a bromine atom into a desired position, subsequently carrying out a Friedel-Crafts reaction with vinyl bromide in the presence of a Lewis acid such as aluminum bromide, aluminum chloride, or iron chloride so as to introduce a 2,2-dibromoethyl group, and subsequently removing HBr using a strong base to thus convert it into an ethynyl group. Specifically, it may be synthesized in accordance with a method described in Macromolecules, 1991, Vol. 24, 5266-5268, 1995, Vol. 28, 5554-5560, Journal of Organic Chemistry, 39, 2995-3003 (1974), etc.

Moreover, it is possible to introduce an alkyl group or a silyl group by making an anion from a hydrogen atom on a terminal acetylene group using butyllithium, etc., and reacting it with an alkyl halide or a silyl halide.

With regard to the compound having a cage structure and/or the polymer having a cage structure, they may be used singly or in a combination of two or more types. Furthermore, the compound having a cage structure and the polymer having a cage structure may be used in combination.

Moreover, the film forming composition of the present invention preferably comprises a compound represented by Formula (1) and a polymer having a cage structure.

When the film forming composition of the present invention comprises a compound having a cage structure and/or a polymer having a cage structure, the total content in the film forming composition of the present invention of the compound represented by Formula (1) and/or the polymer polymerized using at least a compound represented by Formula (1), relative to the total weight of the compound represented by Formula (1), the polymer polymerized using at least a compound represented by Formula (1), the compound having a cage structure, and the polymer having a cage structure, is preferably 5 to 95 wt %, more preferably 5 to 90 wt %, and yet more preferably 10 to 80 wt %.

### Content of Metal

It is preferable for the film forming composition of the present invention to have a sufficiently small content of metal as an impurity. The metal concentration of the film forming composition can be measured at high sensitivity by inductively-coupled plasma mass spectrometry (ICP-MS), and in this case the content of metals other than a transition metal is preferably no greater than 30 ppm, more preferably no greater than 3 ppm, and particularly preferably no greater than 300 ppb.

Furthermore, with regard to the transition metal, from the viewpoint of permittivity of a film obtained in the present invention being increased during pre-baking and thermal curing processes by an oxidation reaction due to high catalytic performance promoting oxidation, the content thereof is preferably as small as possible, and is preferably no greater than 10 ppm, more preferably no greater than 1 ppm, and particularly preferably no greater than 100 ppb.

The metal concentration of the film forming composition of the present invention may be evaluated by subjecting a film obtained using the film forming composition of the present invention to total reflection X-ray fluorescence spectrometry.

When W (tungsten) rays are used as a source of X-rays, K, Ca, Ti, Cr, Mn, Fe, Co, Ni, Cu, Zn, and Pd can be measured as metal elements, and each thereof is preferably no greater than 100×10¹⁰ atom cm⁻², more preferably no greater than 50×10¹⁰ atom cm⁻², and particularly preferably no greater than 10×10¹⁰ atom cm⁻².

It is also possible to measure Br, which is a halogen, and the residual amount thereof is preferably no greater than 10,000×10¹⁰ atom cm⁻², more preferably no greater than 1,000×10¹⁰ atom cm⁻², and particularly preferably no greater than 400×10¹⁰ atom cm⁻². It is also possible to measure Cl as halogen, and from the viewpoint of damage caused to CVD equipment, etching equipment, etc., the residual amount hereof is preferably no greater than 100×10¹⁰ atom cm⁻², more preferably no greater than 50×10¹⁰ atom cm⁻², and particularly preferably no greater than 10×10¹⁰ atom·cm⁻².

### Organic Solvent

The film forming composition of the present invention may comprise an organic solvent.

The organic solvent is not particularly limited, and examples thereof include alcohol-based solvents such as methanol, ethanol, 2-propanol, 1-butanol, 2-ethoxymethanol, 3-methoxypropanol, and 1-methoxy-2-propanol, ketone-based solvents such as acetone, acetylacetone, methyl ethyl ketone, methyl isobutyl ketone, 2-pentanone, 3-pentanone, 2-heptanone, 3-heptanone, cyclopentanone, and cyclohexanone, ester-based solvents such as ethyl acetate, propyl acetate, butyl acetate, isobutyl acetate, pentyl acetate, ethyl propionate, propyl propionate, butyl propionate, isobutyl propionate, propylene glycol monomethyl ether acetate, methyl lactate, ethyl lactate, and γ-butyrolactone, ether-based solvents such as diisopropyl ether, dibutyl ether, ethyl propyl ether, anisole, phenetole, and veratrole, aromatic hydrocarbon-based solvents such as mesitylene, ethylbenzene, diethylbenzene, propylbenzene, and t-butylbenzene, and amide-based solvents such as N-methylpyrrolidinone and dimethylacetamide, and they may be used singly or in a combination of two or more types.

More preferred organic solvents are 1-methoxy-2-propanol, propanol, acetylacetone, cyclohexanone, propylene glycol monomethyl ether acetate, butyl acetate, methyl lactate, ethyl lactate, γ-butyrolactone, anisole, mesitylene, and t-butylbenzene, and particularly preferred organic solvents are 1-methoxy-2-propanol, cyclohexanone, 2-heptanone, propylene glycol monomethyl ether acetate, ethyl lactate, γ-butyrolactone, t-butylbenzene, and anisole.

When an organic solvent is used, the solids content of the film forming composition of the present invention is preferably 1 to 50 wt %, more preferably 2 to 15 wt %, and particularly preferably 3 to 10 wt %.

The polymer having a cage structure preferably has sufficient solubility in an organic solvent. The solubility of the polymer having a cage structure at 25° C. in cyclohexanone or anisole is preferably at least 3 wt %, more preferably at least 5 wt %, and particularly preferably at least 10 wt %.

### Other Additive

Furthermore, an additive such as a radical generator, colloidal silica, a surfactant, a silane coupling agent, or an adhesive may be added to the film forming composition of the present invention in a range that does not impair the properties of an insulating film obtained (heat resistance, permittivity, mechanical strength, coating properties, adhesion, etc.).

As colloidal silica that can be used in the present invention, any colloidal silica may be used. For example, it is a dispersion in which high purity anhydrous silicic acid is dispersed in a hydrophilic organic solvent or water, the average particle size is preferably 5 to 30 nm, and more preferably 10 to 20 nm, and the solids content is 5 to 40 wt %.

As a surfactant that can be used in the present invention, any surfactant may be used. Examples thereof include a nonionic surfactant, an anionic surfactant, and a cationic surfactant, and further examples include a silicone-based surfactant, a fluorine-containing surfactant, a polyalkylene oxide-based surfactant, and an acrylic surfactant. With regard to the surfactant that can be used in the present invention, one type thereof or two or more types may be used. The surfactant is preferably a silicone-based surfactant, a nonionic surfactant, a fluorine-containing surfactant, or an acrylic surfactant, and particularly preferably a silicone-based surfactant.

The amount added of the surfactant that can be used in the present invention, relative to the total amount of the film forming coating solution, is at least 0.01 wt % but no greater than 1 wt %, and more preferably at least 0.1 wt % but no greater than 0.5 wt %.

The silicone-based surfactant referred to in the present invention is a surfactant containing at least one Si atom. As the silicone-based surfactant that can be used in the present invention, any silicone-based surfactant may be used, and a structure containing alkylene oxide and dimethylsiloxane is preferable. It is more preferable for it to be a structure containing the chemical formula below.

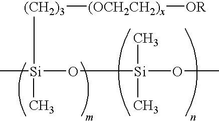

In the formula above, R is a hydrogen atom or an alkyl group having 1 to 5 carbons, x is an integer of 1 to 20, and m and n are independently integers of 2 to 100. Furthermore, where there are plurality of Rs, they may be identical to or different from each other.

Examples of the silicone-based surfactant that can be used in the present invention include BYK-306 and BYK-307 (BYK-Chemie), SH7PA, SH21 PA, SH28PA, and SH30PA (Dow Corning Toray Silicone Co., Ltd.), and Troysol S366 (Troy Chemical Corporation, Inc.).

As a nonionic surfactant that can be used in the present invention, any nonionic surfactant may be used. Examples thereof include polyoxyethylene alkyl ethers, polyoxyethylene aryl ethers, polyoxyethylene dialkyl esters, sorbitan fatty acid esters, fatty acid-modified polyoxyethylenes, and polyoxyethylene-polyoxypropylene block copolymers.

As a fluorine-containing surfactant that can be used in the present invention, any fluorine-containing surfactant may be used. Examples thereof include perfluorooctyl polyethylene oxide, perfluorodecyl polyethylene oxide, and perfluorododecyl polyethylene oxide.

As an acrylic surfactant that can be used in the present invention, any acrylic surfactant may be used. Examples thereof include acrylic acid-based copolymers and methacrylic acid-based copolymers.

As a silane coupling agent that can be used in the present invention, any silane coupling agent may be used.

Examples of the silane coupling agent include 3-glycidyloxypropyltrimethoxysilane, 3-aminoglycidyloxypropyltriethoxysilane, 3-methacryloxypropyltrimethoxysilane, 3-glycidyloxypropylmethyldimethoxysilane, 1-methacryloxypropylmethyldimethoxysilane, 3-aminopropyltrimethoxysilane, 3-aminopropyltriethoxysilane, 2-aminopropyltrimethoxysilane, 2-aminopropyltriethoxysilane, N-(2-aminoethyl)-3-aminopropyltrimethoxysilane, N-(2-aminoethyl)-3-aminopropylmethyldimethoxysilane, 3-ureidopropyltrimethoxysilane, 3-ureidopropyltriethoxysilane, N-ethoxycarbonyl-3-aminopropyltrimethoxysilane, N-ethoxycarbonyl-3-aminopropyltriethoxysilane, N-triethoxysilylpropyltriethylenetriamine, N-triethoxysilylpropyltriethylenetriamine, 10-trimethoxysilyl-1,4,7-triazadecane, 10-triethoxysilyl-1,4,7-triazadecane, 9-trimethoxysilyl-3,6-diazanonyl acetate, 9-triethoxysilyl-3,6-diazanonyl acetate, N-benzyl-3-aminopropyltrimethoxysilane, N-benzyl-3-aminopropyltriethoxysilane, N-phenyl-3-aminopropyltrimethoxysilane, N-phenyl-3-aminopropyltriethoxysilane, N-bis(oxyethylene)-3-aminopropyltrimethoxysilane, and N-bis(oxyethylene)-3-aminopropyltriethoxysilane.

With regard to the silane coupling agent that can be used in the present invention, one type thereof may be used on its own, or two or more types may be used in combination.

As an adhesion promoter that can be used in the present invention, any adhesion promoter may be used.

Examples of the adhesion promoter include trimethoxysilylbenzoic acid, γ-methacryloxypropyltrimethoxysilane, vinyltriacetoxysilane, vinyltrimethoxysilane, γ-isocyanatopropyltriethoxysilane, γ-glycidoxypropyltrimethoxysilane, β-(3,4-epoxycyclohexyl)ethyltrimethoxysilane, trimethoxyvinylsilane, γ-aminopropyltriethoxysilane, aluminum monoethylacetoacetate diisopropionate, vinyltris(2-methoxyethoxy)silane, N-(2-aminoethyl)-3-aminopropylmethyldimethoxysilane, N-(2-aminoethyl)-3-aminopropyltrimethoxysilane, 3-chloropropylmethyldimethoxysilane, 3-chloropropyltrimethoxysilane, 3-methacryloxypropyltrimethoxysilane, 3-mercaptopropyltrimethoxysilane, trimethylchlorosilane, dimethylvinylchlorosilane, methyldiphenylchlorosilane, chloromethyldimethylchlorosilane, trimethylmethoxysilane, dimethyldiethoxysilane, methyldimethoxysilane, dimethylvinylethoxysilane, diphenyldimethoxysilane, phenyltriethoxysilane, hexamethyldisilazane, N,N′-bis(trimethylsilyl)urea, dimethyltrimethylsilylamine, trimethylsilylimidazole, vinyltrichlorosilane, benzotriazole, benzimidazole, indazole, imidazole, 2-mercaptobenzimidazole, 2-mercaptobenzothiazole, 2-mercaptobenzoxazole, urazole, thiouracil, mercaptoimidazole, mercaptopyrimidine, 1,1-dimethylurea, 1,3-dimethylurea, and a thiourea compound. A functionalized silane coupling agent is preferable as the adhesion promoter.

The amount of adhesion promoter used, relative to 100 parts by weight of the total solids content, is preferably no greater than 10 parts by weight, and is particularly preferably 0.05 to 5 parts by weight.

The film forming composition of the present invention may employ a pore forming factor in a range that allows the film to have mechanical strength, thus making the film porous and giving low permittivity.

The pore forming factor as an additive that becomes a pore forming agent is not particularly limited, but a non-metallic compound is suitably used, and it is necessary to simultaneously satisfy solubility in a solvent that is used in a film forming coating solution and compatibility with the polymer of the present invention.

Furthermore, the boiling point or decomposition temperature of the pore forming agent is preferably 100° C. to 500° C., more preferably 200° C. to 450° C., and particularly preferably 250° C. to 400° C.

The molecular weight is preferably 200 to 50,000, more preferably 300 to 10,000, and particularly preferably 400 to 5,000.

The amount thereof added, relative to the polymer forming a film, is preferably 0.5 to 75 wt %, more preferably 0.5 to 30 wt %, and particularly preferably 1 to 20 wt %.

Furthermore, as the pore forming factor, the polymer may contain a decomposable group, and the decomposition temperature thereof is preferably 100° C. to 500° C., more preferably 200° C. to 450° C., and particularly preferably 250° C. to 400° C. The content of the decomposable group, relative to the polymer forming a film, is preferably 0.5 to 75 mole %, more preferably 0.5 to 30 mole %, and particularly preferably 1 to 20 mole %.

### Film

The film of the present invention is a film obtained using the film forming composition of the present invention, and may be used suitably as an insulating film.

Furthermore, a process for producing the film of the present invention is not particularly limited, but preferably comprises a step of preparing the film forming composition of the present invention, a step of applying the film forming composition of the present invention in the form of a film, and a step of heating the film thus applied.

A film obtained using the film forming composition of the present invention may be formed by coating a substrate with the film forming composition by any method such as a spin coating method, a roller coating method, a dip coating method, or a scan method, and removing the solvent by a heating treatment. As the method for coating the substrate, the spin coating method and the scan method are preferable. The spin coating method is particularly preferable. For spin coating, commercial equipment may be used. Preferred examples thereof include the Clean Track Series (Tokyo Electron Ltd.), the D-Spin Series (Dainippon Screen Manufacturing Co., Ltd.), and the SS Series or CS Series (Tokyo Ohka Kogyo Co., Ltd.). With regard to conditions for spin coating, any rotational speed may be employed, but from the viewpoint of in-plane uniformity of the film the rotational speed is preferably on the order of 1,300 rpm for a 300 mm silicon substrate.

Furthermore, a method for discharging the film forming composition may be either dynamic discharge in which a composition solution is discharged onto a rotating substrate or static discharge in which a composition solution is discharged onto a stationary substrate, and from the viewpoint of in-plane uniformity of the film, dynamic discharge is preferable. Furthermore, from the viewpoint of suppressing consumption of the composition, it is possible to employ a method in which after a liquid film is formed by preliminarily discharging only a main solvent of the composition onto a substrate, the composition is discharged thereonto. The spin coating time is not particularly limited, but from the viewpoint of throughput it is preferably within 180 sec. Furthermore, from the viewpoint of a substrate being transported, it is preferable to carry out a treatment for preventing film from being left on the edge of the substrate (edge rinse, back rinse).

A method for the heating treatment is not particularly limited, and hot plate heating, a heating method using a furnace, light irradiation heating using a xenon lamp by an RTP (Rapid Thermal Processor), etc., which are generally used, may be used. A heating method employing hot plate heating or a furnace is preferable. As a hot plate, commercial equipment may be preferably used, and the Clean Track Series (Tokyo Electron Ltd.), the D-Spin Series (Dainippon Screen Manufacturing Co., Ltd.), the SS Series or CS Series (Tokyo Ohka Kogyo Co., Ltd.), etc. may be preferably used. As a furnace, the α series (Tokyo Electron Ltd.), etc. may be preferably used.

The film forming composition of the present invention is particularly preferably cured by a heating treatment after coating a substrate therewith. For example, a polymerization reaction when post-heating carbon triple bonds etc. remaining in the film formed by the film forming composition may be utilized. The conditions for this post-heating treatment are preferably 100° C. to 450° C., more preferably 200° C. to 420° C., and particularly preferably 350° C. to 400° C., and are preferably in the range of 1 min to 2 hours, more preferably 10 min to 1.5 hours, and particularly preferably 30 min to 1 hour. The post-heating treatment may be carried out a plurality of times. Furthermore, this post heating is particularly preferably carried out under a nitrogen atmosphere in order to prevent thermal oxidation by oxygen.

Furthermore, in the present invention, instead of the heating treatment, a polymerization reaction of carbon triple bonds etc. remaining in the polymer may be effected by irradiating with a high-energy beam, thus carrying out curing. Examples of the high-energy beam include an electron beam, UV rays, and X-rays, but are not particularly limited to these methods.

When an electron beam is used as the high energy beam, the energy is preferably 0 to 50 keV, more preferably 0 to 30 keV, and particularly preferably 0 to 20 keV. The total dose of the electron beam is preferably 0 to 5 μC/cm², more preferably 0 to 2 μC/cm², and particularly preferably 0 to 1 μC/cm². The substrate temperature when irradiating with an electron beam is preferably 0° C. to 450° C., more preferably 0° C. to 400° C., and particularly preferably 0° C. to 350° C. The pressure is preferably 0 to 133 kPa, more preferably 0 to 60 kPa, and particularly preferably 0 to 20 kPa. From the viewpoint of preventing oxidation of the polymer, the atmosphere surrounding the substrate is preferably an inert atmosphere such as Ar, He, or nitrogen. Furthermore, a gas such as oxygen, a hydrocarbon, or ammonia may be added for the purpose of a reaction with a plasma, an electromagnetic wave, or a chemical species generated by interaction with the electron beam. Irradiation with an electron beam in the present invention may be carried out a plurality of times, and in this case the conditions for irradiation with the electron beam need not be the same each time, and different conditions may be employed each time.

As a high energy beam, UV rays may be used. The irradiation wavelength region when UV rays are used is preferably 190 to 400 nm, and the output thereof is preferably 0.1 to 2,000 mWcm⁻² immediately above the substrate. The substrate temperature when irradiated with UV rays is preferably 250° C. to 450° C., more preferably 250° C. to 400° C., and particularly preferably 250° C. to 350° C. From the viewpoint of preventing oxidation of the polymer, the atmosphere surrounding the substrate is preferably an inert atmosphere such as Ar, He, or nitrogen. In this case, the pressure is preferably 0 to 133 kPa.

A film obtained using the film forming composition of the present invention may be suitably used as an insulating film, and more suitably as an interlayer insulating film for a semiconductor. That is, an insulating film obtained using the film forming composition of the present invention may be used suitably in an electronic device.

For example, when used as an interlayer insulating film for a semiconductor, in the wiring structure, a barrier layer for preventing metal migration may be provided on the wiring side face, furthermore, a cap layer, an interlayer adhesion layer, an etching stopper layer, etc. for preventing peeling off in CMP (Chemical Mechanical Polishing) may be provided on upper and lower faces of the wiring or the interlayer insulating film and, moreover, a layer of the interlayer insulating film may be divided into a plurality of layers using another type of material as necessary.

A film obtained using the film forming composition of the present invention may be subjected to an etching process for copper wiring or for another purpose. With regard to etching, either wet etching or dry etching may be employed, and dry etching is preferable. Dry etching may employ either an ammonia-based plasma or a fluorocarbon-based plasma as appropriate. These plasmas may employ not only Ar but also a gas such as oxygen, nitrogen, hydrogen, or helium. After etching, ashing may be carried out in order to remove a photoresist, etc. used for etching, and washing may be carried out in order to remove a residue after ashing.

A film obtained using the film forming composition of the present invention may be subjected to CMP after a copper wiring process in order to planarize a copper plated portion. As a CMP slurry (liquid reagent), a commercial slurry (e.g. those manufactured by Fujimi Inc., Rodel-Nitta, JSR Corporation, Hitachi Chemical Ltd., etc.) may be used as appropriate. As CMP equipment, commercial equipment (Applied Materials, Inc., Ebara Corporation, etc.) may be used as appropriate. Furthermore, in order to remove a slurry residue after CMP, washing may be carried out.

A film obtained using the film forming composition of the present invention may be used for various purposes. For example, it is suitable as an insulating film in a semiconductor device such as an LSI, a system LSI, a DRAM, an SDRAM, an RDRAM, or a D-RDRAM or an electronic component such as a multichip module multilayer wiring board, and it may be used as an interlayer insulating film for a semiconductor, an etching stopper film, a surface protecting film, a buffer coat film and, furthermore, as a passivation film in an LSI, an α-ray shielding film, a coverlay film for a flexible printed board, an overcoat film, a cover coat for a flexible copper-clad board, a solder resist film, a liquid crystal orientation film, etc.

Furthermore, in another intended application, the film of the present invention is doped with an electron donor or acceptor so as to impart electrical conductivity thereto, and may be used as an electrically conductive film.

A film obtained using the film forming composition of the present invention preferably has a desired specific permittivity according to an intended application, but the specific permittivity is preferably low, and the film is more preferably an insulating film.

As a method for measuring the specific permittivity of a film obtained by using the film forming composition of the present invention, a known method may be used, and it is preferable to calculate it from a capacitance value at 1 MHz using a mercury probe manufactured by Four Dimensions Inc. and an HP4285ALCR meter manufactured by Yokogawa Hewlett-Packard Company.

Furthermore, a film obtained using the film forming composition of the present invention is a film having excellent mechanical strength, and preferably has a high Young's modulus.

As a method for measuring the Young's modulus of a film obtained by using the film forming composition of the present invention, a known method may be used, and it is preferable to measure it using an SA2 Nanoindentor from MTS.

In accordance with the present invention, there can be provided a film forming composition that enables a film having excellent heat resistance and mechanical strength, low permittivity, and good film surface condition to be formed, an insulating film obtained using the film forming composition, and an electronic device comprising the insulating film.

### EXAMPLES

The Examples below explain the present invention, but should not be construed as limiting the scope thereof.

### Synthetic Example 1

### Synthesis of Compound (a)

Compound (a) was synthesized in accordance with the scheme below.

In the scheme, ‘eq.’ denotes molar equivalents, and ‘%’ denotes yield. The same applies to the schemes in Synthetic Examples 2 to 8, which are described later. In the schemes below, Ph denotes a phenyl group, i-Pr denotes an isopropyl group, THF denotes tetrahydrofuran, DMSO denotes dimethyl sulfoxide, TMS denotes a trimethylsilyl group, and Me denotes a methyl group.

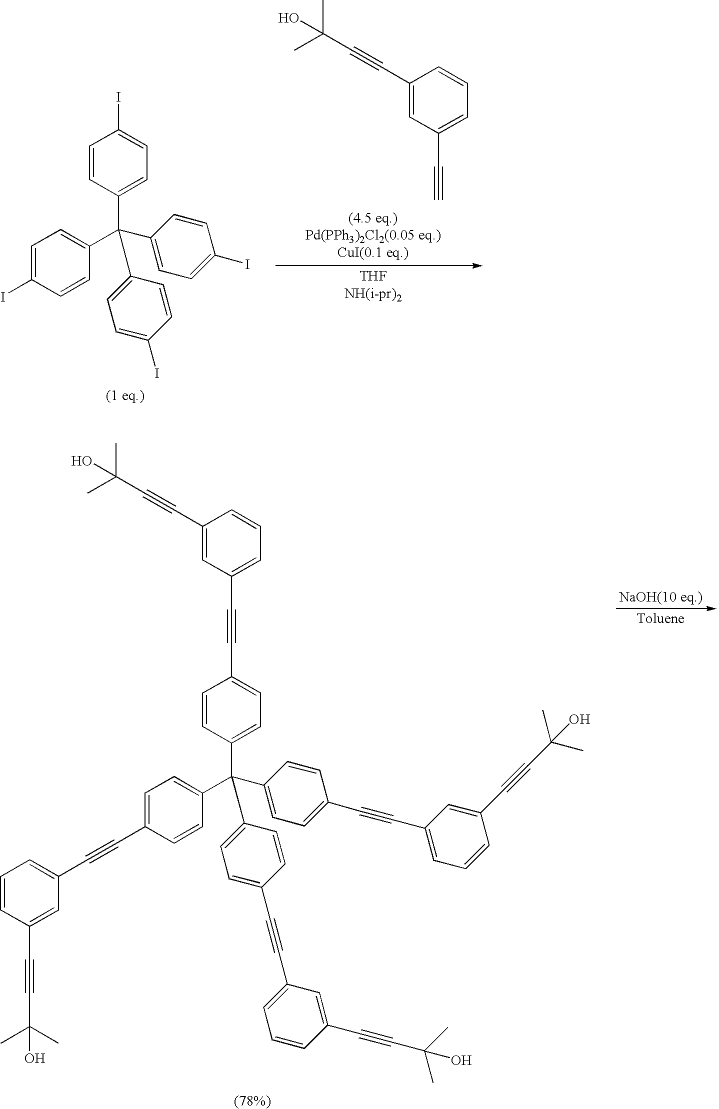

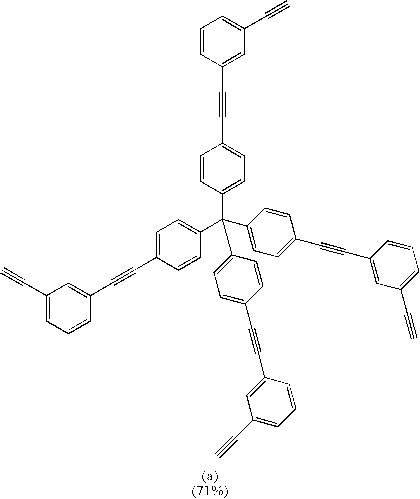

### Synthetic Example 2

### Synthesis of Compound (b)

Compound (b) was synthesized in accordance with the scheme below.

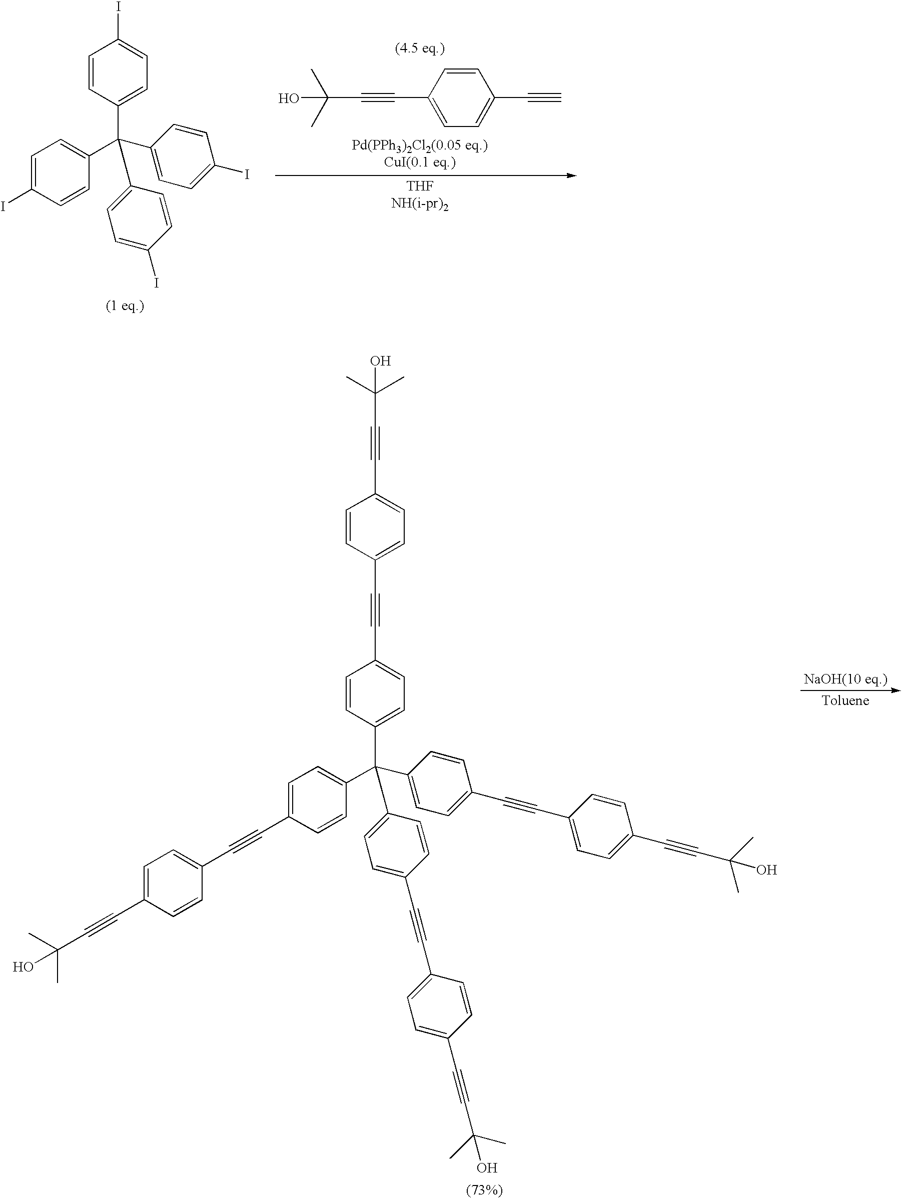

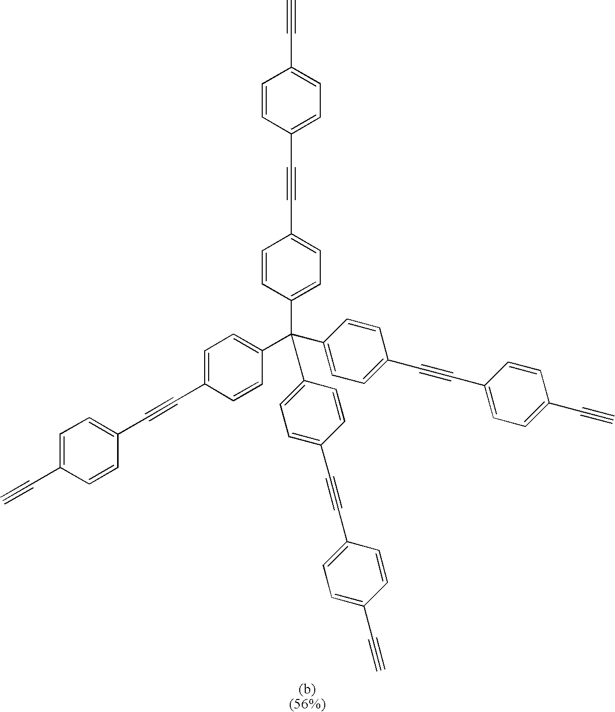

### Synthetic Example 3

### Synthesis of Compound (c)

Compound (c) was synthesized in accordance with the scheme below.

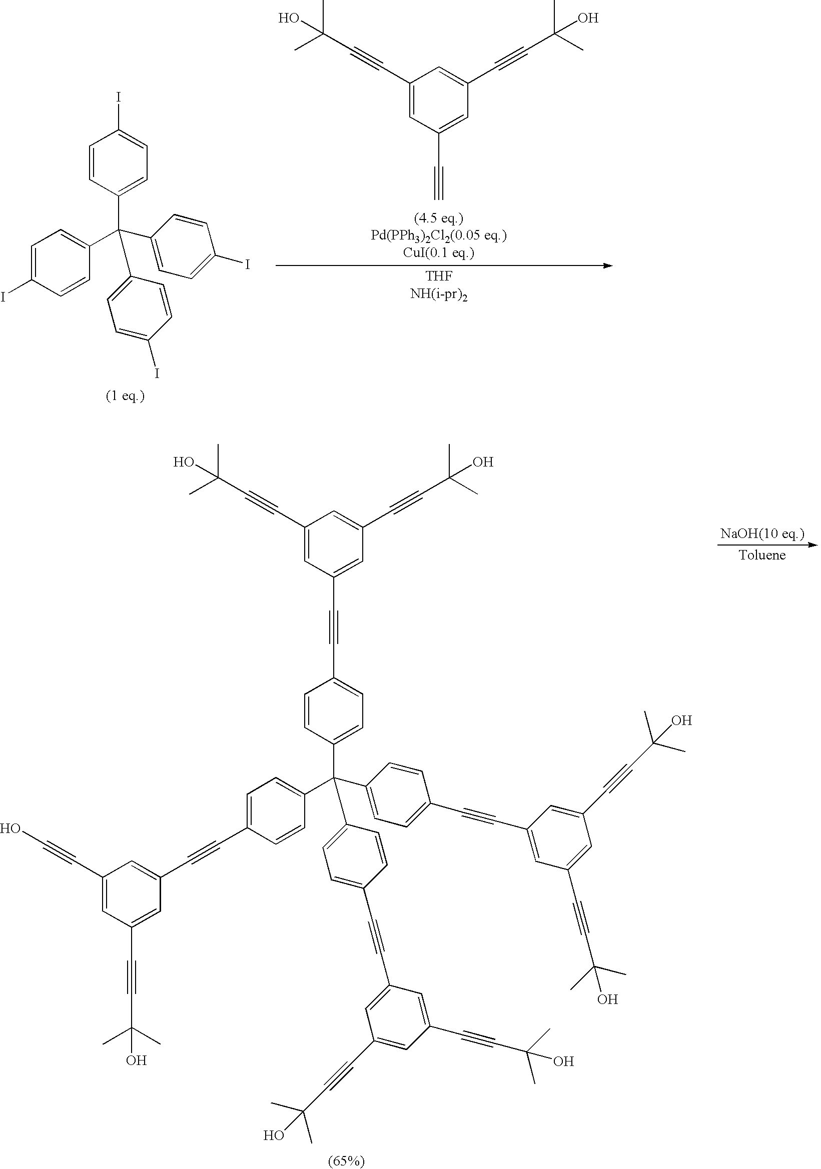

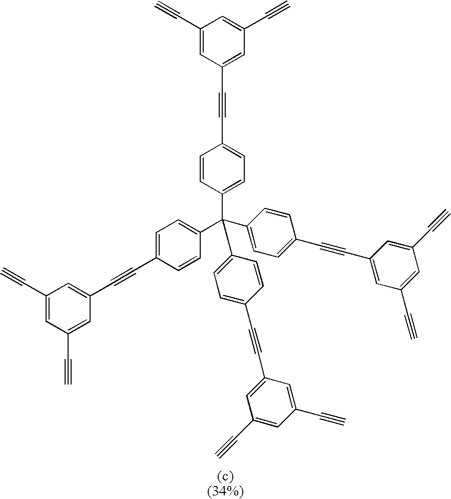

### Synthetic Example 4

### Synthesis of Compound (d)

Compound (d) was synthesized in accordance with the scheme below.

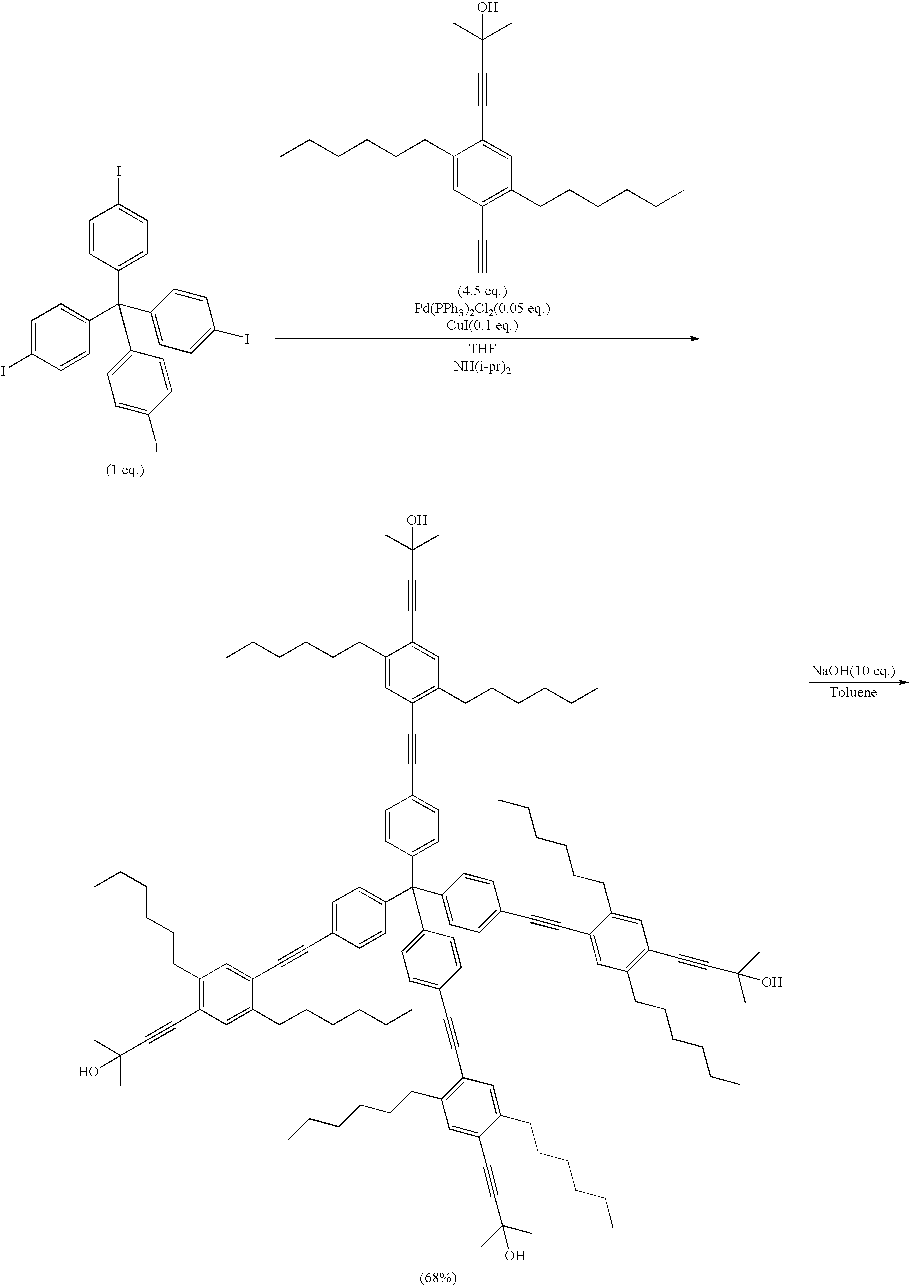

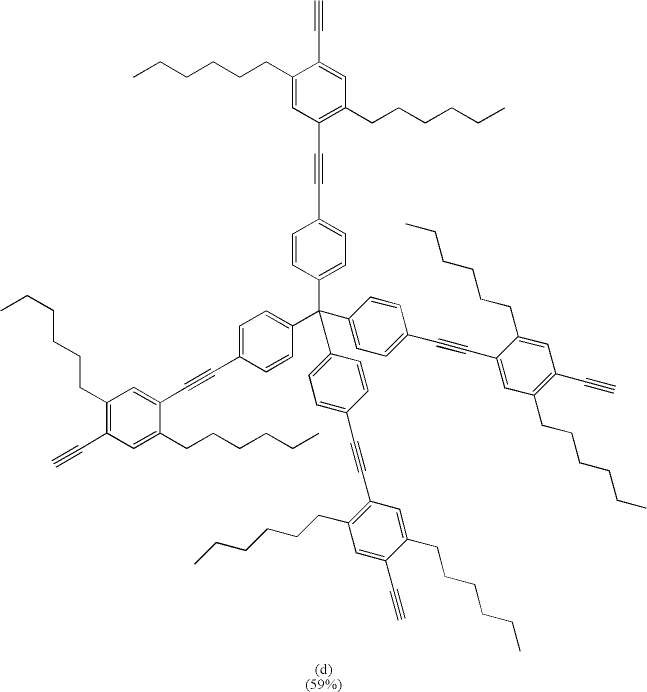

### Synthetic Example 5

### Synthesis of Compound (e)

Compound (e) was synthesized in accordance with the scheme below.

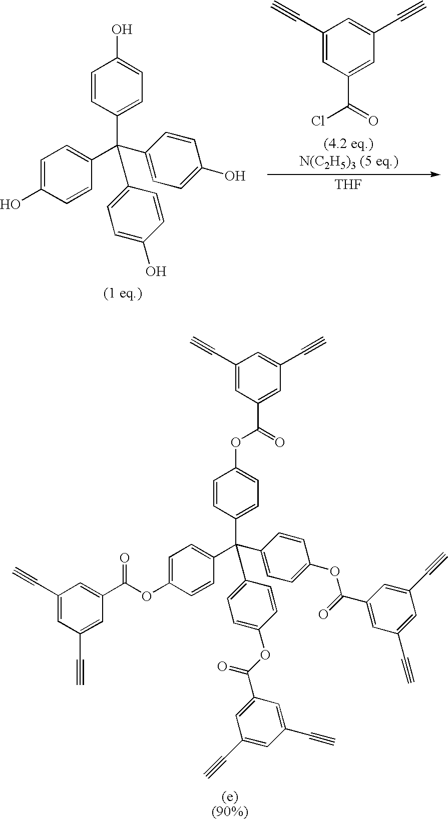

### Synthetic Example 6

### Synthesis of Compound (f)

Compound (f) was synthesized in accordance with the scheme below.

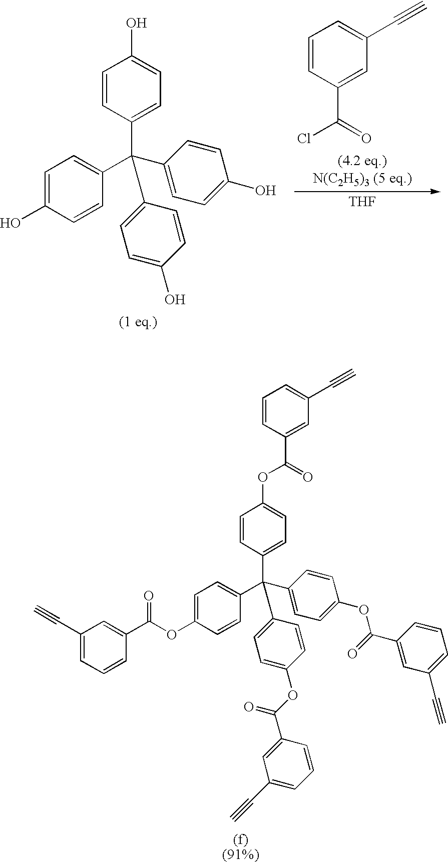

### Synthetic Example 7

### Synthesis of Compound (q)

Compound (g) was synthesized in accordance with the scheme below.

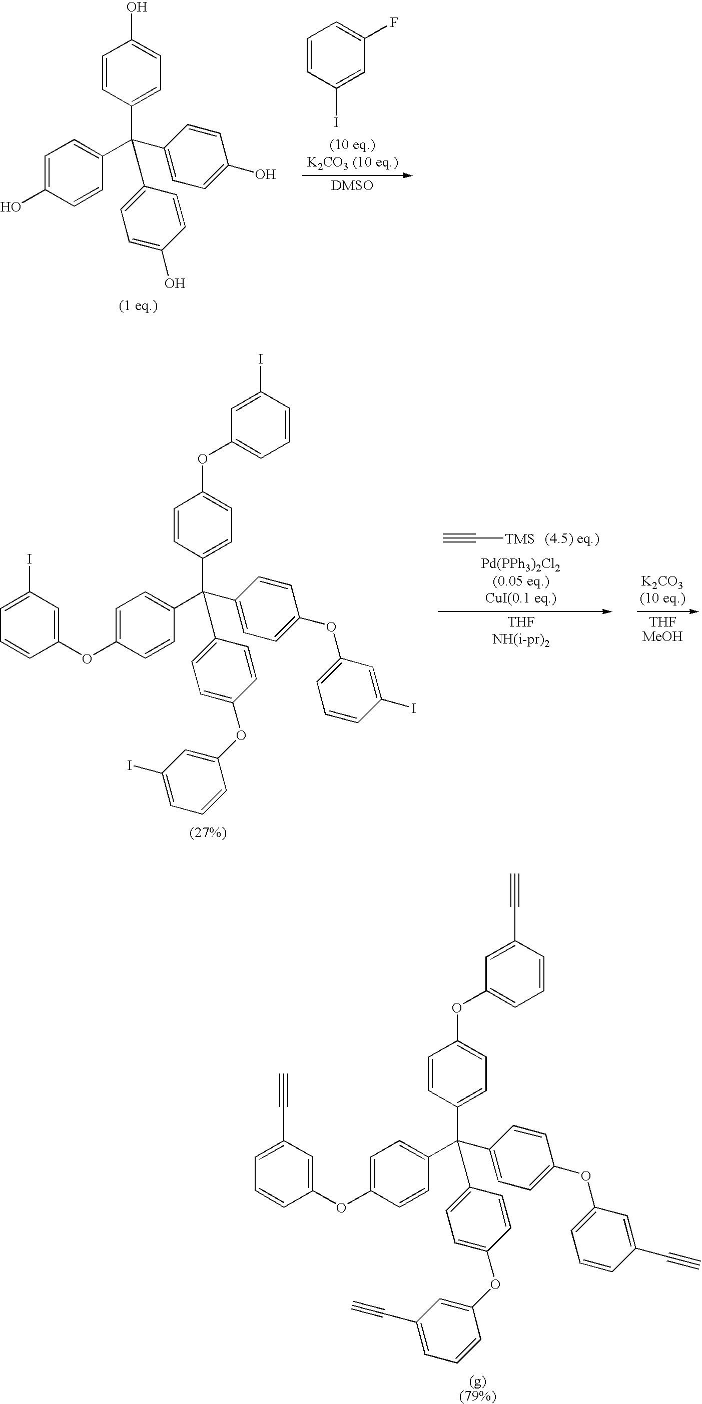

### Synthetic Example 8

### Synthesis of Compound (h)

Compound (h) was synthesized in accordance with the scheme below.

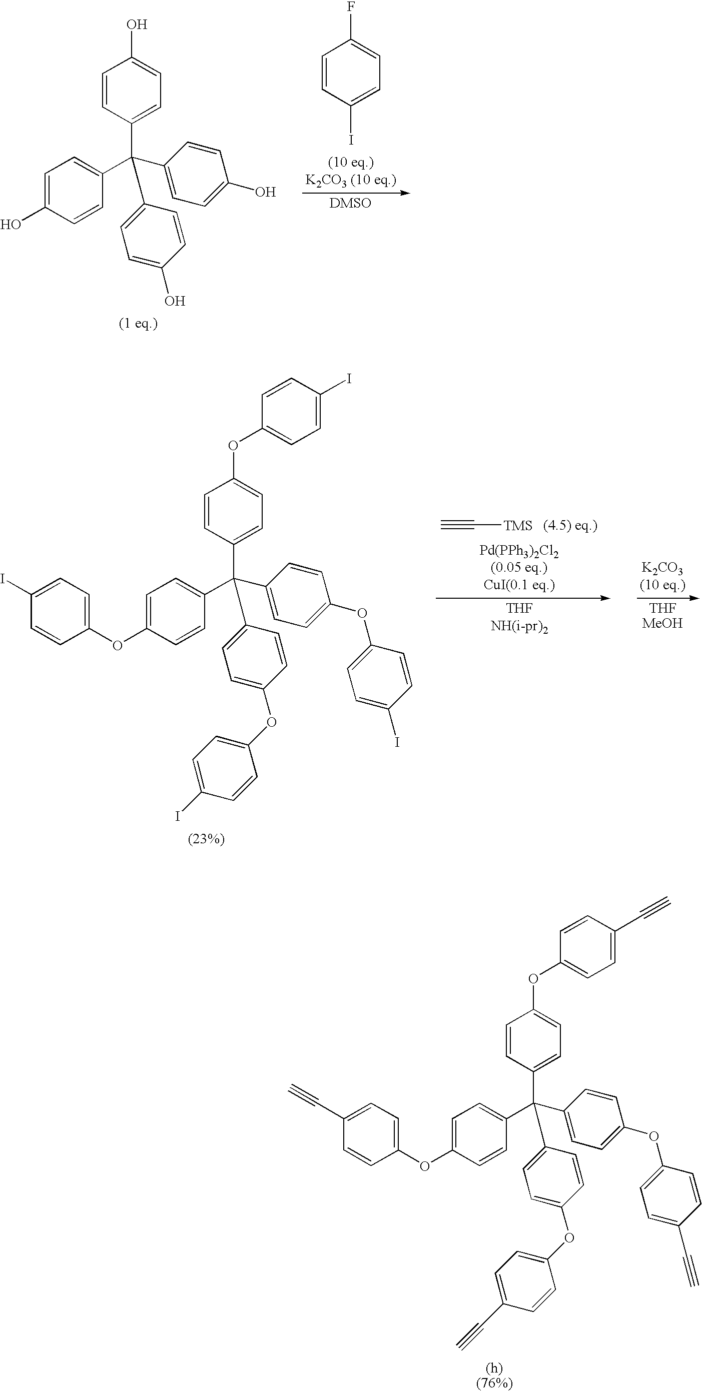

### Synthetic Example 9

### Synthesis of Polymer (1)

5 parts by weight of compound (a) and 95 parts by weight of t-butylbenzene were placed in a reaction vessel and heated to an internal temperature of 120° C. while stirring under a flow of nitrogen, thus completely dissolving compound (a). Subsequently, a solution of 2.2 parts by weight of dicumyl peroxide (PERCUMYL D, from NOF Corporation) in 1.9 parts by weight of diphenyl ether was added dropwise to the reaction mixture over 1 hour while keeping the internal temperature of the reaction mixture at 120° C. to 130° C.

After the reaction, the reaction mixture was cooled to 50° C., 316 parts by weight of methanol was then added thereto, and a solid thus precipitated was filtered and washed with methanol. After vacuum drying, 2.2 parts by weight of polymer (1) having a weight-average molecular weight of about 90,000 was obtained.

### Synthetic Example 10

### Synthesis of Polymer (2)

5 parts by weight of compound (b) and 95 parts by weight of t-butylbenzene were placed in a reaction vessel and heated to an internal temperature of 120° C. while stirring under a flow of nitrogen, thus completely dissolving compound (b). Subsequently, a solution of 2.2 parts by weight of dicumyl peroxide (PERCUMYL D, from NOF Corporation) in 1.9 parts by weight of diphenyl ether was added dropwise to the reaction mixture over 1 hour while keeping the internal temperature of the reaction mixture at 120° C. to 130° C.

After the reaction, the reaction mixture was cooled to 50° C., 316 parts by weight of methanol was then added thereto, and a solid thus precipitated was filtered and washed with methanol. After vacuum drying, 2.0 parts by weight of polymer (2) having a weight-average molecular weight of about 73,000 was obtained.

### Synthetic Example 11

### Synthesis of Polymer (3)

5 parts by weight of compound (g) and 95 parts by weight of t-butylbenzene were placed in a reaction vessel and heated to an internal temperature of 120° C. while stirring under a flow of nitrogen, thus completely dissolving compound (g). Subsequently, a solution of 2.2 parts by weight of dicumyl peroxide (PERCUMYL D, from NOF Corporation) in 1.9 parts by weight of diphenyl ether was added dropwise to the reaction mixture over 1 hour while keeping the internal temperature of the reaction mixture at 120° C. to 130° C.

After the reaction, the reaction mixture was cooled to 50° C., 316 parts by weight of methanol was then added thereto, and a solid thus precipitated was filtered and washed with methanol. After vacuum drying, 1.5 parts by weight of polymer (3) having a weight-average molecular weight of about 73,000 was obtained.

### Synthetic Example 12

### Synthesis of Polymer (4)

1,3,5-Triethynyladamantane was synthesized in accordance with a synthetic method described in J. Polym. Sci. PART A Polym. Chem., Vol. 30, p.1747 (1992).

10 parts by weight of 1,3,5-triethynyladamantane and 56 parts by weight of t-butylbenzene were placed in a reaction vessel and heated to an internal temperature of 120° C. while stirring under a flow of nitrogen, thus completely dissolving the 1,3,5-triethynyladamantane. Subsequently, a solution of 2.2 parts by weight of dicumyl peroxide (PERCUMYL D, from NOF Corporation) in 1.9 parts by weight of diphenyl ether was added dropwise to the reaction mixture over 1 hour while keeping the internal temperature of the reaction mixture at 120° C. to 130° C.

After the reaction, the reaction mixture was cooled to 50° C., 314 parts by weight of 2-propanol was then added thereto, and a solid thus precipitated was filtered and washed with 2-propanol. Purification by reprecipitation was carried out by dissolving a polymer thus obtained in 35.6 parts by weight of tetrahydrofuran (THF) and adding it to 316 parts by weight of methanol. After vacuum drying, 4.2 parts by weight of polymer (4) having a weight-average molecular weight of about 60,000 was obtained.

### Synthetic Example 13

### Synthesis of Polymer (5)

10 parts by weight of 1,3,5-trivinyladamantane and 56 parts by weight of t-butylbenzene were placed in a reaction vessel and heated to an internal temperature of 120° C. while stirring under a flow of nitrogen, thus completely dissolving the 1,3,5-trivinyladamantane. Subsequently, a solution of 2.2 parts by weight of dicumyl peroxide (PERCUMYL D, from NOF Corporation) in 1.9 parts by weight of diphenyl ether was added dropwise to the reaction mixture over 1 hour while keeping the internal temperature of the reaction mixture at 120° C. to 130° C.

After the reaction, the reaction mixture was cooled to 50° C., 314 parts by weight of 2-propanol was then added thereto, and a solid thus precipitated was filtered and washed with 2-propanol. Purification by reprecipitation was carried out by dissolving a polymer thus obtained in 35.6 parts by weight of THF and adding it to 316 parts by weight of methanol. After vacuum drying, 4.0 parts by weight of polymer (5) having a weight-average molecular weight of about 55,000 was obtained.

### Example 1

A coating solution was prepared by completely dissolving 1.0 part by weight of polymer (1) alone in 9.0 parts by weight of cyclohexanone. The coating obtained was heated on a hot plate under a flow of nitrogen at 250° C. for 60 sec and further calcined in a nitrogen-flushed oven at 400° C. for 60 minutes, and a 0.5 μm thick film was obtained.

When the specific permittivity of the film (measurement temperature: 25° C., the same applies below) was calculated from a capacitance value at 1 MHz using a mercury probe manufactured by Four Dimensions Inc. and an HP4285ALCR meter manufactured by Yokogawa Hewlett-Packard Company, it was 2.80. Furthermore, when the Young's modulus was measured using an SA2 Nanoindentor from MTS, it was 14.0 GPa.

### Example 2

A coating solution was prepared by completely dissolving 1.0 part by weight of polymer (2) alone in 9.0 parts by weight of cyclohexanone. The coating obtained was heated on a hot plate under a flow of nitrogen at 250° C. for 60 sec and further calcined in a nitrogen-flushed oven at 400° C. for 60 minutes, and a 0.5 μm thick film was obtained.

When the specific permittivity of the film (measurement temperature: 25° C., the same applies below) was calculated from a capacitance value at 1 MHz using a mercury probe manufactured by Four Dimensions Inc. and an HP4285ALCR meter manufactured by Yokogawa Hewlett-Packard Company, it was 2.79. Furthermore, when the Young's modulus was measured using an SA2 Nanoindentor from MTS, it was 12.3 GPa.

### Example 3

A coating solution was prepared by completely dissolving 1.0 part by weight of polymer (3) alone in 9.0 parts by weight of cyclohexanone. The coating obtained was heated on a hot plate under a flow of nitrogen at 250° C. for 60 sec and further calcined in a nitrogen-flushed oven at 400° C. for 60 minutes, and a 0.5 μm thick film was obtained.

When the specific permittivity of the film (measurement temperature: 25° C., the same applies below) was calculated from a capacitance value at 1 MHz using a mercury probe manufactured by Four Dimensions Inc. and an HP4285ALCR meter manufactured by Yokogawa Hewlett-Packard Company, it was 2.83. Furthermore, when the Young's modulus was measured using an SA2 Nanoindentor from MTS, it was 8.0 GPa.

### Comparative Example 1

A coating solution was prepared by completely dissolving 1.0 part by weight of polymer (4) alone in 9.0 parts by weight of cyclohexanone. The coating obtained was heated on a hot plate under a flow of nitrogen at 250° C. for 60 sec and further calcined in a nitrogen-flushed oven at 400° C. for 60 minutes, and a 0.5 μm thick film was obtained.

When the specific permittivity of the film (measurement temperature: 25° C., the same applies below) was calculated from a capacitance value at 1 MHz using a mercury probe manufactured by Four Dimensions Inc. and an HP4285ALCR meter manufactured by Yokogawa Hewlett-Packard Company, it was 2.65. Furthermore, when the Young's modulus was measured using an SA2 Nanoindentor from MTS, it was 5.0 GPa.

### Comparative Example 2

A coating solution was prepared by completely dissolving 1.0 part by weight of polymer (5) alone in 9.0 parts by weight of cyclohexanone. The coating obtained was heated on a hot plate under a flow of nitrogen at 250° C. for 60 sec and further calcined in a nitrogen-flushed oven at 400° C. for 60 minutes, and a 0.5 μm thick film was obtained.

When the specific permittivity of the film (measurement temperature: 25° C., the same applies below) was calculated from a capacitance value at 1 MHz using a mercury probe manufactured by Four Dimensions Inc. and an HP4285ALCR meter manufactured by Yokogawa Hewlett-Packard Company, it was 2.63. Furthermore, when the Young's modulus was measured using an SA2 Nanoindentor from MTS, it was 3.5 GPa.

### Example 4

Coating solutions were prepared by completely dissolving 1.0 part by weight in total of polymer (4) and each of compounds (a) to (h) and polymers (1) to (3) obtained in Synthetic Examples 1-11 in 9.0 parts by weight of cyclohexanone. These solutions were filtered using a tetrafluoroethylene filter having a pore size of 0.1 μm, silicon wafers were spin-coated therewith, these coatings were heated on a hot plate under a flow of nitrogen at 250° C. for 60 sec and further calcined in a nitrogen-flushed oven at 400° C. for 60 minutes, and 0.5 μm thick uniform films free of particulates were obtained.

The specific permittivity of the films (measurement temperature: 25° C., the same applies below) was calculated from a capacitance value at 1 MHz using a mercury probe manufactured by Four Dimensions Inc. and an HP4285ALCR meter manufactured by Yokogawa Hewlett-Packard Company. The appearance of the insulating films obtained was examined with a pocket microloupe (50 times) manufactured by Peak Optics, and there were no cracks on the surface of the coatings. Furthermore, the Young's modulus was measured using an SA2 Nanoindentor from MTS.

The results are given in Table 2 below.

### Example 5

Coating solutions were prepared by completely dissolving 1.0 part by weight in total of polymer (5) and each of compounds (a) to (h) and polymers (1) to (3) obtained in Synthetic Examples 1-11 in 9.0 parts by weight of cyclohexanone. These solutions were filtered using a tetrafluoroethylene filter having a pore size of 0.1 μm, silicon wafers were spin-coated therewith, these coatings were heated on a hot plate under a flow of nitrogen at 250° C. for 60 sec and further calcined in a nitrogen-flushed oven at 400° C. for 60 minutes, and 0.5 μm thick films were obtained.

The specific permittivity of the films (measurement temperature: 25° C., the same applies below) was calculated from a capacitance value at 1 MHz using a mercury probe manufactured by Four Dimensions Inc. and an HP4285ALCR meter manufactured by Yokogawa Hewlett-Packard Company. Furthermore, the Young's modulus was measured using an SA2 Nanoindentor from MTS.

The results are given in Table 3 below.

### Comparison of Heat Resistance and Film Surface Condition

Evaluation of the heat resistance was carried out by heating in air at 400° C. for 30 sec and measuring a change in weight between that before and that after heating.

The film surface condition was examined using an OLYMPUS MX50. When cloudiness as shown in FIG. 1 was observed, the surface condition was evaluated as poor, when a uniform film as shown in FIG. 2 was observed, the surface condition was evaluated as good, and when cloudiness was observed in part of the film, the surface condition was evaluated as fair.

TABLE 1

| | | | | | Percentage | |
|---|---|---|---|---|---|---|
| | | | | | weight loss after | |
| | | Amount | | Young's | heating in air at | Film |
| Film | | added | Initial k | modulus | 400° C. for 30 sec. | surface |
| material | Additive | (wt %) | value | (GPa) | (%) | condition |
| Polymer (1) | None | — | 2.80 | 14.0 | 0.3 | Good |
| Polymer (2) | None | — | 2.79 | 12.3 | 0.5 | Good |
| Polymer (3) | None | — | 2.83 | 8.0 | 1.5 | Good |

TABLE 2

| | | Amount | | | | |
|---|---|---|---|---|---|---|
| | | of | | | Percentage weight | |
| | | Additive | | Young's | loss after heating | Film |
| Film | | added | Initial | modulus | in air at 400° C. for | surface |
| material | Additive | (wt %) | k value | (GPa) | 30 sec. (%) | condition |
| Polymer | — | — | 2.65 | 5.0 | 15.0 | Good |
| (4) | Compound | 30 | 2.75 | 8.6 | 5.7 | Good |
| | (a) | 50 | 2.78 | 10.3 | 1.6 | Good |
| | Compound | 20 | 2.74 | 7.1 | 6.5 | Good |
| | (b) | 50 | 2.77 | 9.6 | 1.9 | Good |
| | Compound | 20 | 2.77 | 9.9 | 5.0 | Good |
| | (c) | 50 | 2.81 | 11.3 | 1.5 | Good |
| | Compound | 20 | 2.68 | 6.5 | 7.5 | Good |
| | (d) | 50 | 2.72 | 7.7 | 5.1 | Good |
| | Compound | 30 | 2.82 | 5.6 | 7.1 | Good |
| | (e) | 50 | 2.85 | 6.4 | 3.7 | Fair |
| | Compound | 30 | 2.78 | 5.8 | 8.2 | Good |
| | (f) | 50 | 2.82 | 6.3 | 4.5 | Fair |
| | Compound | 20 | 2.77 | 5.3 | 8.6 | Good |
| | (g) | 60 | 2.81 | 6.4 | 4.5 | Good |
| | Compound | 20 | 2.78 | 5.4 | 9.9 | Good |
| | (h) | 50 | 2.82 | 6.6 | 6.7 | Good |
| | Polymer (1) | 30 | 2.70 | 9.2 | 1.5 | Good |
| | Polymer (2) | 30 | 2.69 | 8.9 | 1.9 | Good |
| | Polymer (3) | 30 | 2.72 | 5.9 | 3.2 | Good |

TABLE 3

| | | Amount | | | | |
|---|---|---|---|---|---|---|
| | | of | | | Percentage weight | |
| | | Additive | | Young's | loss after heating | Film |
| Film | | added | Initial | modulus | in air at 400° C. for | surface |
| material | Additive | (wt %) | k value | (GPa) | 30 sec. (%) | condition |
| Polymer | — | — | 2.63 | 3.5 | 18.4 | Good |
| (5) | Compound | 30 | 2.73 | 5.6 | 8.7 | Good |
| | (a) | 50 | 2.76 | 7.3 | 2.7 | Good |
| | Compound | 20 | 2.72 | 5.4 | 8.9 | Good |
| | (b) | 50 | 2.75 | 7.1 | 3.1 | Good |
| | Compound | 20 | 2.75 | 6.8 | 7.5 | Good |
| | (c) | 50 | 2.79 | 9.1 | 2.4 | Good |
| | Compound | 20 | 2.66 | 4.9 | 9.2 | Good |
| | (d) | 50 | 2.70 | 6.1 | 5.8 | Good |
| | Compound | 30 | 2.81 | 5.3 | 8.3 | Good |
| | (e) | 50 | 2.84 | 6.2 | 4.2 | Fair |
| | Compound | 30 | 2.78 | 4.1 | 10.3 | Good |
| | (f) | 50 | 2.82 | 5.0 | 8.2 | Fair |
| | Compound | 20 | 2.75 | 3.8 | 9.8 | Good |
| | (g) | 60 | 2.79 | 4.5 | 5.4 | Good |
| | Compound | 20 | 2.78 | 3.8 | 10.1 | Good |
| | (h) | 50 | 2.82 | 4.6 | 6.1 | Good |
| | Polymer (1) | 30 | 2.69 | 7.8 | 2.4 | Good |
| | Polymer (2) | 30 | 2.68 | 7.4 | 2.5 | Good |
| | Polymer (3) | 30 | 2.71 | 5.1 | 4.9 | Good |

It has been found that an insulating film formed using the film forming composition of the present invention exhibits excellent heat resistance and mechanical strength, low permittivity, and good film surface condition. Furthermore, it has been found that when a polymer of a compound represented by Formula (1) is added, the permittivity is lower than a case in which the same amount of the compound represented by Formula (1) is added.

What is claimed is:

1. A film forming composition comprising a compound represented by Formula (1) below and/or a polymer polymerized using at least a compound represented by Formula (1) below, 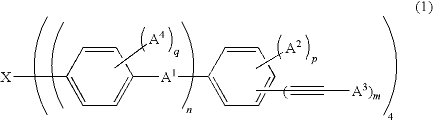
wherein each A¹ independently denotes a single bond, —CO—, —NR—, —O—, —SO₂—, —CH═CH—, —C≡C—, or a group in which two or more of the above are combined, A² and A⁴ independently denote a monovalent organic group having 1 to 10 carbons, each A³ independently denotes a hydrogen atom or a monovalent organic group having 1 to 10 carbons but excluding a phenyl group, X denotes a tetravalent organic group having 1 to 9 carbons, R denotes a hydrogen atom or an alkyl group having 1 to 10 carbons, each m independently denotes an integer of 1 to 5, each n independently denotes an integer of 1 or more, each p independently denotes an integer of 0 to 4, and each q independently denotes an integer of 0 to 4.

2. The film forming composition according to claim 1, wherein each A¹ in Formula (1) above independently denotes —CO—O—, —O—CO—, —O—, —CH═CH—, or —C≡C—.

3. The film forming composition according to claim 1, wherein X in Formula (1) above is a carbon atom.

4. The film forming composition according to claim 1, wherein all of the four groups bonding to X in Formula (1) above are the same groups.

5. The film forming composition according to claim 1, wherein the compound represented by Formula (1) above is a compound selected from the group consisting of compound (a) to compound (h) below.

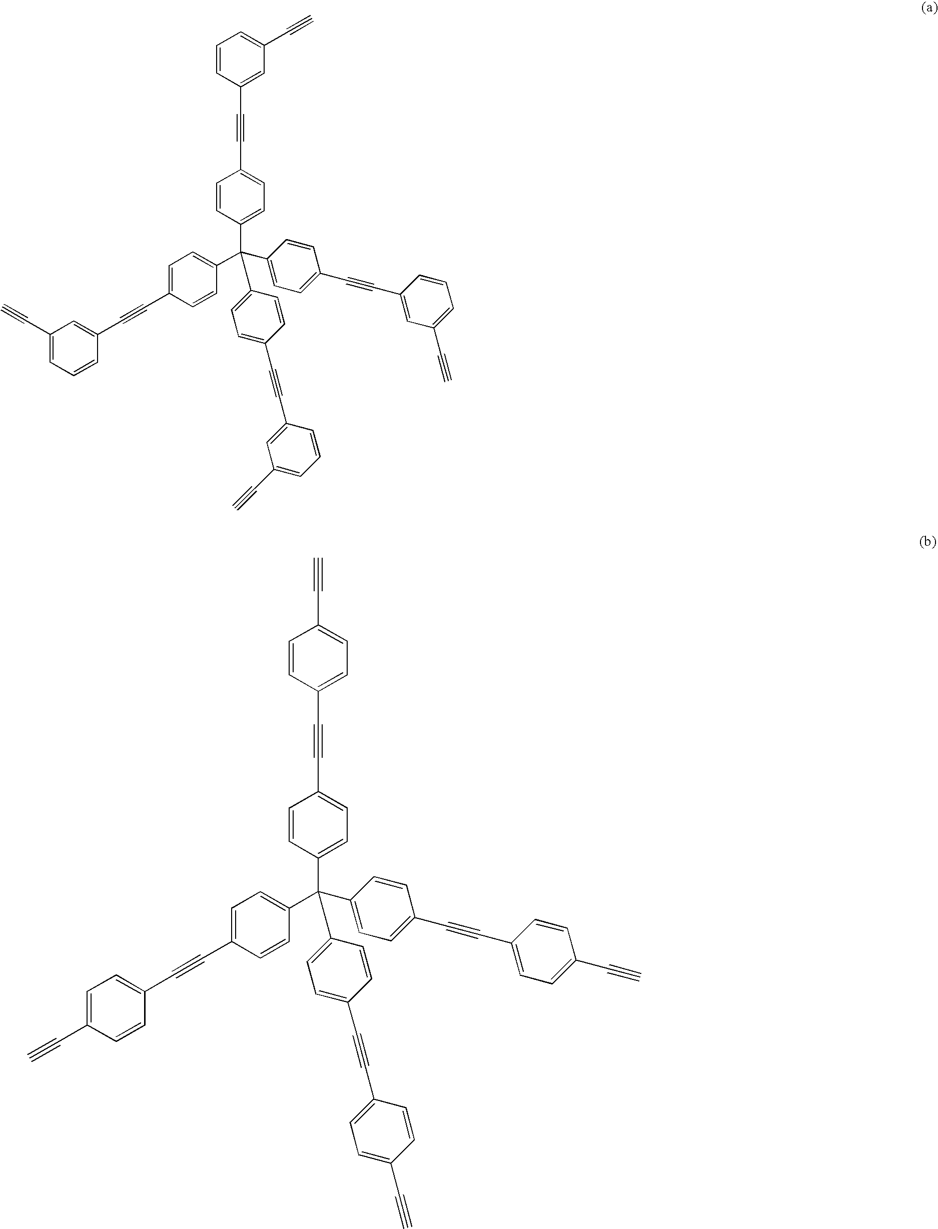

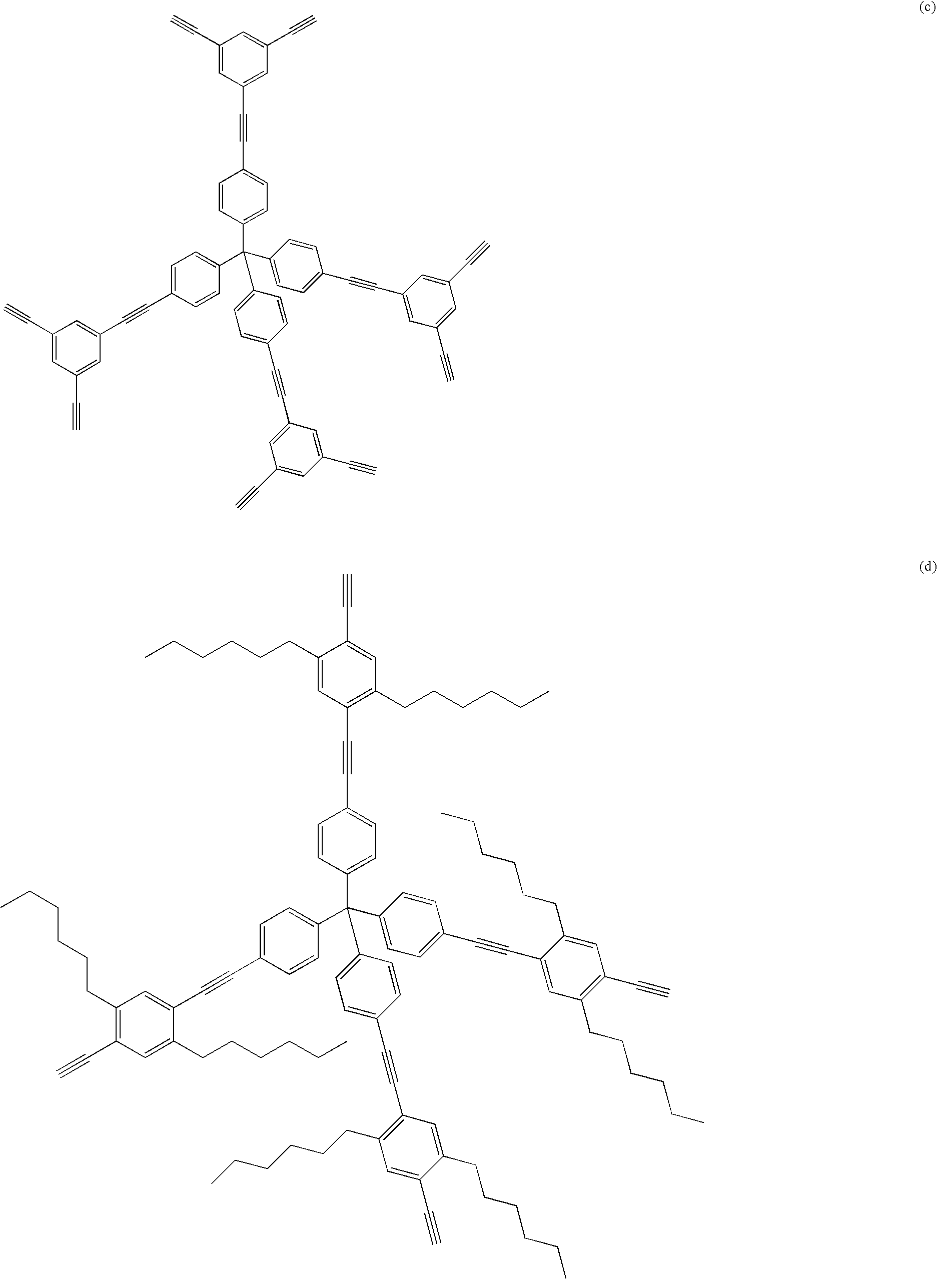

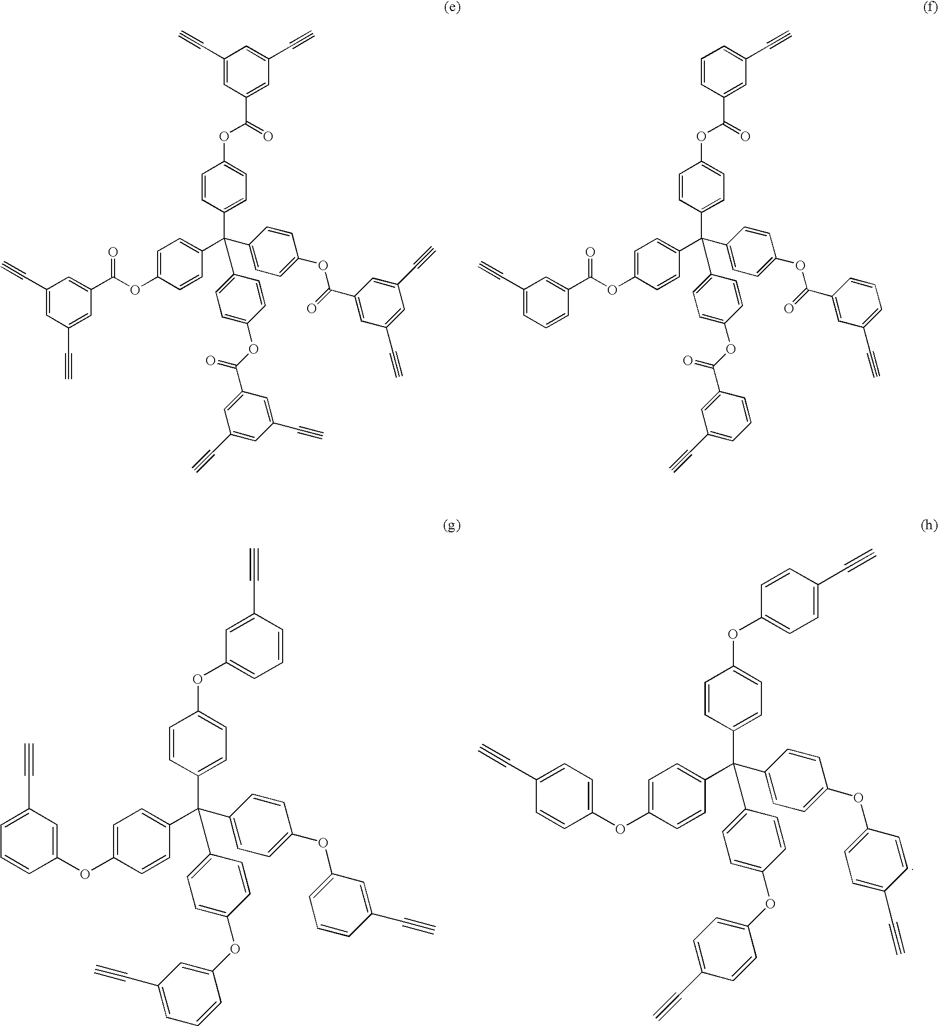

6. The film forming composition according to claim 1, wherein it comprises a polymer obtained by thermally polymerizing or by polymerizing in the presence of a radical initiator or in the presence of a transition metal catalyst a monomer represented by Formula (1) above.

7. The film forming composition according to claim 1, wherein it comprises a compound having a cage structure and/or a polymer having a cage structure.

8. The film forming composition according to claim 1, wherein it comprises a polymer having a cage structure.

9. The film forming composition according to claim 1, wherein it comprises a polymer having a cage structure obtained by polymerizing a monomer having a cage structure in the presence of a radical initiator or in the presence of a transition metal catalyst.

10. The film forming composition according to claim 9, wherein the monomer having a cage structure has a polymerizable carbon-carbon double bond and/or carbon-carbon triple bond.

11. The film forming composition according to claim 7, wherein the cage structure is a structure selected from the group consisting of adamantane, biadamantane, diamantane, triamantane, and tetramantane.

12. The film forming composition according to claim 9, wherein the monomer having a cage structure is a monomer selected from the group consisting of monomers represented by Formulae (2) to (7) below, 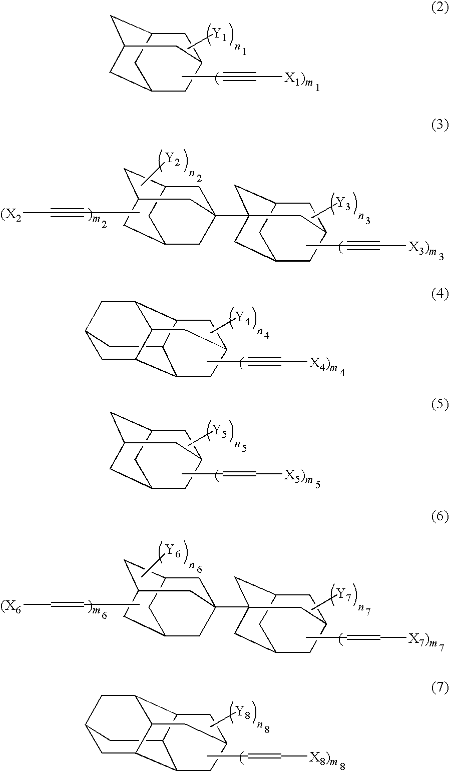
wherein X₁ to X₈ independently denote a hydrogen atom, an alkyl group, an alkenyl group, an alkynyl group, an aryl group, a silyl group, an acyl group, an alkoxycarbonyl group, or a carbamoyl group, Y₁ to Y₈ independently denote a halogen atom, an alkyl group, an aryl group, or a silyl group, m₁ and m₅ denote an integer of 1 to 16, n₁ and n₅ denote an integer of 0 to 15, m₂, m₃, m₆, and m₇ independently denote an integer of 1 to 15, n₂, n₃, n₆, and n₇ denote an integer of 0 to 14, m₄ and m₈ denote an integer of 1 to 20, and n₄ and n₈ denote an integer of 0 to 19.

13. A film obtained using the film forming composition according to claim 1.

14. The film according to claim 13, wherein it is an insulating film.

15. An electronic device comprising the film according to claim 13.
